(19) United States
(12) Patent Application Publication
(10) Pub. No.: US 2013/0224257 A1
(43) Pub. Date: Aug. 29, 2013

(54) METHOD FOR PREPARING MICROSPHERES AND MICROSPHERES PRODUCED THEREBY

(71) Applicants: SK Chemicals Co., Ltd., (US); Ewha University - Industry Collaboration Foundation, (US)

(72) Inventors: Hong Kee Sah, Seoul (KR); Bong-Yong Lee, Seoul (KR); Key-An Um, Suwon (KR); Joon-Gyo Oh, Suwon (KR); Yong Youn Hwang, Suwon (KR); Hong-Kee Kim, Gunpo (KR); Kyu Ho Lee, Suwon (KR); Seok Hyun Hong, Seoul (KR); Yoon-Jung Lee, Yongin (KR)

(73) Assignees: Ewha University - Industry Collaboration Foundation, Seoul (KR); SK Chemicals Co., Ltd., Seongnam (KR)

(21) Appl. No.: 13/745,075

(22) Filed: Jan. 18, 2013

Related U.S. Application Data
(63) Continuation of application No. PCT/KR2011/005347, filed on Jul. 20, 2011.

(30) Foreign Application Priority Data
Jul. 21, 2010 (KR) 10-2010-0070407

(51) Int. Cl. A61K 9/16; A61K 31/551; A61K 31/4196

(52) U.S. Cl. CPC A61K 9/1694; A61K 31/4196; A61K 31/551; A61K 9/16
USPC 424/400; 514/383; 514/220

(57) ABSTRACT

The present invention relates to a method for preparing microsphere and microspheres produced thereby. The method comprises: mixing a water-insoluble organic solvent with a dispersion solvent; mixing a polymer compound, a drug and a water-insoluble organic solvent to prepare a dispersed phase; mixing the dispersed phase with the dispersion solvent mixed with the water-insoluble organic solvent to prepare an emulsion; and adding a base or an acid to the prepared emulsion. With the method, it is possible to prepare a drug-containing polymeric microsphere cost-effectively and conveniently.

### CROSS-REFERENCE TO RELATED APPLICATIONS

This application is a continuation of International Application No. PCT/KR2011/005347 filed Jul. 20, 2011, which claims priority to Korean Application No. 10-2010-0070407 filed on Jul. 21, 2010, which applications are incorporated herein by reference.

### TECHNICAL FIELD

The present invention relates to a method for preparing microsphere and microspheres produced by thereby. More particularly, the present invention relates to a method for preparing a polymeric microsphere comprising the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an emulsion; and adding a base or an acid to the prepared emulsion so as to remove the water-insoluble organic solvent from the emulsion and a polymeric microsphere thereby, and a composition for drug delivery comprising the microspheres.

### BACKGROUND ART

Conventional injectable formulations such as solution, suspension, and emulsion are quickly removed from the body after administration, and therefore frequent administration is essentially needed for treatment of chronic diseases. Microencapsulation has been developed to solve the problem, and referred to a production process for encapsulating drugs in microspheres (hereinafter, the term microsphere will include nanospheres) consisting of high molecular compounds. Microspheres are usually in a size of μm unit, and can be administered to a human or animal by intramuscular or subcutaneous injection. Further, microspheres can be produced to have a variety of drug release rates, so that the period of drug delivery can be controlled. Therefore, even if a therapeutic drug is administered only once, its effective concentration can be maintained over a long period of time, and the total administration amount of therapeutic drug can be minimized to improve the drug compliance in patients. Accordingly, world famous pharmaceutical companies are very interested in the production of polymeric microsphere loaded with drugs.

In the production of polymeric microspheres by microencapsulation, poly-d,l-lactide-co-glycolide (PLGA) has been most widely used as a high molecular compound. PLGA is a biocompatible high molecular compound that is hydrolyzed in vivo to be converted into nontoxic lactic acid and glycolic acid. Therefore, pharmaceutical industries have made extensive studies on the development of drug formulation using PLGA, and examples of current available microsphere product produced by using PLGA include Risperdal Consta, Sandostatin LAR, Vivitrol, and Lupron Depot. Each of them is administered to a patient once to control the release of risperidone, octreotide acetate, naltrexone, and leuprolide acetate from 2 weeks to 4 months.

Such polymeric microspheres loaded with drugs can be conventionally produced by a solvent evaporation method or a solvent extraction method using an organic solvent such as methylene chloride and ethyl acetate.

First, the solvent evaporation method will be briefly described (see U.S. Pat. Nos. 6,471,996, 5,985,309, and 5,271,945). A drug is dispersed or dissolved in an organic solvent in which a high molecular compound is dissolved, and then emulsified in a dispersion medium such as water to produce an oil-in-water (O/W) emulsion. Then the organic solvent in the emulsion is diffused into a dispersion medium and evaporated across the air/water interface to form the polymeric microspheres loaded with drugs. At this time, in order to accelerate the diffusion of organic solvent into the dispersion medium, a method such as organic solvent extraction using reduced pressure, increased temperature, and an excessive amount of water is used. A dispersion organic solvent that is generally used to dissolve the high molecular PLGA is methylene chloride, which dissolves a PLGA copolymer well using various molecular weights and lactide:glycolide ratios and because it does not mix well with water due to the low water solubility of 1.32% by weight. Thus, methylene chloride is a suitable solvent for the production of oil-in-water emulsion. Further, due to the low boiling point of 39.8° C., a small amount of methylene chloride molecules that diffused from emulsion liquid droplets into water are evaporated across the water/air interface. Such process is continuously repeated to remove methylene chloride from emulsion droplets, thereby forming microspheres. Finally, the residual methylene chloride present in microspheres is easily dried and removed due to its low boiling point.

Likewise, even though methylene chloride is the most optimal solvent used for the production of emulsion in that it is very volatile, not mixed well with water, and has a lower boiling point than water, methylene chloride has the following problems: (a) it is a carcinogen proved by experiments; (b) it destroys the ozone layer in the atmosphere to generate a toxic environment, causing an increase in human skin cancer; (c) it is one of the 38 toxic and hazardous substances announced by the agency for toxic substances and disease registry within the US Department of Health and Human Services; (d) a lot of time is required to completely remove methylene chloride in the emulsion droplets, since it has a low water solubility of about 1.32% by weight and only a small amount of methylene chloride is dissolved in water and evaporates. For example, in U.S. Pat. No. 6,884,435, the emulsion is stirred overnight to remove methylene chloride from the emulsion, and conditions such as increased temperature or reduced pressure in a reactor are introduced to shorten the production time of microspheres (see U.S. Pat. Nos. 3,691,090, 3,891,570, 6,270,700, and 6,572,894).

On the other hand, the solvent extraction method used to produce polymeric microspheres loaded with drugs is a method for effectively extracting the organic solvent in the emulsion droplets by using a large amount of solubilizing solvent. When the organic solvent is extracted from the emulsion droplets, the dissolved high molecular compounds are hardened to convert the emulsion droplets into microspheres. The solubilizing solvent that is generally used is water, and the degree of water solubility of the organic solvent greatly affects the amount of water needed. For example, methylene chloride has water solubility of 1.32% by weight, whereby a very large amount of water is needed for extracting methylene chloride in the emulsion. However, a large amount of wastewater containing methylene chloride is produced, in which the treatment of the wastewater becomes a problematic issue. Therefore, in the solvent extraction method, ethyl acetate, which has higher water solubility than methylene chloride, is mainly used. Since ethyl acetate has the water solubility of 8.7% by weight, it can be extracted by using a relatively small amount of water, as compared to methylene chloride, and it is advantageously a nonhalogenated organic solvent. However, its boiling point is 77° C. and much higher than 39.8° C., which is that of methylene chloride. Thus, ethyl acetate has a drawback that the residual solvent is hard to remove when dried. Furthermore, a high molecular PLGA compound with a specific molecular weight and lactide:glycolide ratio has a characteristic of not dissolving easily in ethyl acetate.

Therefore, technologies simultaneously employing the solvent evaporation method and solvent extraction method are disclosed in U.S. Pat. Nos. 4,389,840, 4,530,840, 6,544,559, 6,368,632, and 6,572,894. That is, in the methods, the emulsion is produced, and then the organic solvent is partially removed by the evaporation process, and the residual organic solvent is removed by the solvent extraction method. For example, U.S. Pat. No. 4,389,840 discloses a method for producing microspheres, in which a drug and a high molecular PLGA are dissolved in methylene chloride and then emulsified in water to produce oil in water-type emulsion, then 40 to 60% by weight of methylene chloride is removed by the evaporation process, and the residual methylene chloride is extracted using a large amount of water to produce microspheres.

However, since all of the organic solvents used in the known methods do not have sufficient high water solubility, excessively large amounts of water (over 10 times more than water solubility of organic solvent) should be used. Thus, a large-volume reactor is needed, and a large amount of wastewater containing organic solvent is produced, as a result, the cost for wastewater treatment is increased. Further, there is a problem that the residual organic solvent present in the microspheres is not effectively removed.

The present inventors disclosed an method for preparing microspheres containing a drug comprising the step of changing insoluble organic solvent to soluble organic solvent by adding ammonium solution in Korean Patent No. 10-0918092. Through the method, a microspheres could be prepared easily and quickly with minimizing generation of waste water. However, this method has still a problem that the amount of remaining organic solvent is over 1%.

In particular, when a large amount of organic solvent remains in the microspheres, the microspheres tend to coalesce during the drying process. As a result, since the microspheres may not be dispersed separately after the drying process, a problem may occur during injection and the reproducibility of drug release may decrease. Further, if the amount of the remaining solvent exceeds an allowable limit, it will be difficult to get the regulatory approval.

Therefore, the development of novel method minimizing organic solvent remains in microspheres is urgent.

### SUMMARY

Accordingly, the inventors of the present invention have conducted research on a method for reducing the amount of organic solvent remaining in a prepared polymeric microsphere, in a polymeric microsphere preparation method including a removal step of a water-insoluble organic solvent by an acid or base. As a result, they found that when a water-insoluble organic solvent used in dispersed phase preparation is previously added to a dispersion solvent, the concentration of organic solvent remaining in the prepared polymeric microsphere is further reduced. Then, they completed the present invention based on the findings.

Accordingly, an object of the present invention is to provide a method for preparing a polymeric microsphere, the method including the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an O/W (oil-in-water), O/O (oil-in-oil) or W/O/W (water-in oil-in-water) type emulsion; and adding a base or an acid to the prepared emulsion so as to remove the water-insoluble organic solvent from the emulsion.

Another object of the present invention is to provide a method for preparing a polymeric microsphere, the method including the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an O/W (oil-in-water), O/O (oil-in-oil) or W/O/W (water-in oil-in-water) type emulsion; adding a base or an acid to the prepared emulsion so as to remove the water-insoluble organic solvent from the emulsion; and obtaining the prepared polymeric microsphere whose water-insoluble organic solvent is removed and re-dispersing the obtained polymeric microsphere in a warmed dispersion solvent.

A further object of the present invention is to provide a polymeric microsphere prepared by the above method.

A still further object of the present invention is to provide a drug delivery composition including, as an active ingredient, a polymeric microsphere prepared by the above method.

In accordance with an aspect of the present invention, there is provided a method for preparing a polymeric microsphere, the method including the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an O/W (oil-in-water), O/O (oil-in-oil) or W/O/W (water-in oil-in-water) type emulsion; and adding a base or an acid to the prepared emulsion so as to remove the water-insoluble organic solvent from the emulsion.

In accordance with another aspect of the present invention, there is provided a method for preparing a polymeric microsphere, the method including the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an O/W (oil-in-water), O/O (oil-in-oil) or W/O/W (water-in oil-in-water) type emulsion; adding a base or an acid to the prepared emulsion so as to remove the water-insoluble organic solvent from the emulsion; and obtaining the prepared polymeric microsphere whose water-insoluble organic solvent is removed and re-dispersing the obtained polymeric microsphere in a warmed dispersion solvent.

In accordance with a further aspect of the present invention, there is provided a polymeric microsphere prepared by the above method.

In accordance with a still further object of the present invention, there is provided a drug delivery composition including, as an active ingredient, a polymeric microsphere prepared by the above method.

As described above, the present invention provides a novel method for preparing a polymeric microsphere, the polymeric microsphere prepared by the method, and a drug delivery composition containing the polymeric microsphere, in which the method includes the steps of: mixing a water-insoluble organic solvent with a dispersion solvent; and removing the water-insoluble organic solvent from an emulsion by base or acid. In the inventive preparation method, it is possible to conveniently prepare a target drug-containing polymeric microsphere within a short time by using a small amount of water, with the generation of a minimized amount of waste solution without a requirement of a conventional solvent-evaporation or solvent-extraction process. Also, since a low concentration of remaining solvent within a prepared polymeric microsphere can be maintained, it is effective in preparation in sustained-release type medicines.

### BRIEF DESCRIPTION OF DRAWINGS

FIG. 1 shows a graph showing the measurement result of a remaining solvent of a polymeric microsphere prepared without previous addition of a water-insoluble organic solvent to a dispersion solvent;

FIG. 2 shows an electron microscopic photograph of an olanzapine-containing polymeric microsphere, in which FIG. 2A shows a polymeric microsphere prepared by maintaining the temperature in a preparation process at 33° C., and FIG. 2B shows a polymeric microsphere prepared without a warming step, in which in both cases, it can be found that the polymeric microsphere was well prepared in a spherical shape, and olanzapine was well loaded into the polymeric microsphere;

FIG. 3 shows an electron microscopic photograph of a polymeric microsphere prepared by maintaining the temperature in the second step at 40° C., and increasing the amounts of a drug and a polymer compound, in which FIG. 3A shows a polymeric microsphere in a case where the amount of a drug is 60 mg, and FIG. 3B shows a polymeric microsphere in a case where the amount of a drug is 80 mg;

FIG. 4 shows a graph showing the concentration of a remaining solvent within a polymeric microsphere according to the concentration of an organic solvent previously added to a dispersion solvent;

FIG. 5 shows an electron microscopic photograph of a polymeric microsphere prepared by varying the concentration of an organic solvent previously added to a dispersion solvent (Add: amount (ml) of a water-insoluble organic solvent (ethyl formate) previously added to a dispersion solvent);

FIG. 6 shows a standard curve for measuring the molecular weight of a polymer compound before/after the polymeric microsphere preparation;

FIG. 7 shows an electron microscopic photograph of an olanzapine-containing polymeric microsphere (preparation no. is for an olanzapine-containing polymeric microsphere in Example);

FIG. 8 shows a graph showing the measurement result of a concentration change of olanzapine in the blood when a composition containing an olanzapine-containing polymeric microsphere was injected into a rat (preparation no. is for an olanzapine-containing polymeric microsphere in Example 9);

FIG. 9 shows an electron microscopic photograph of an anastrozole-containing polymeric microsphere (the number in the photograph indicates the preparation no. in Example 10, and the anastrozole-containing polymeric microsphere was prepared according to the composition of the corresponding preparation number);

FIG. 10 shows a graph showing the result of in vitro release test of an anastrozole-containing polymeric microsphere prepared by the inventive method; and

FIG. 11 shows a graph showing the result of an animal test on the persistence of an anastrozole-containing polymeric microsphere prepared by the inventive method, in which the anastrozole-containing polymeric microsphere with each composition, prepared by the inventive method, was intramuscularly injected into a rat (in an amount of 20 mg/kg), and the concentration of anastrozole in blood was measured.

### DETAILED DESCRIPTION

Hereinafter, the present invention will be described in more detail. Embodiments of the present invention provide methods for preparing a polymeric microsphere, which include the steps of: (a) mixing a water-insoluble organic solvent with a dispersion solvent; (b) mixing a polymer compound, a drug and a water-insoluble organic solvent so as to prepare a dispersed phase; (c) mixing the dispersed phase in the step (b) with the dispersion solvent mixed with the water-insoluble organic solvent in the step (a) so as to prepare an O/W (oil-in-water), O/O (oil-in-oil) or W/O/W (water-in oil-in-water) type emulsion; and (d) adding a base or an acid to the prepared emulsion in the step (c) so as to remove the water-insoluble organic solvent from the emulsion.

In the step (a), a water-insoluble organic solvent and a dispersion solvent are mixed. The water-insoluble organic solvent used in the present invention may be used without limitation if it is well known in the art, as long as it is capable of dissolving the polymer which is used for the preparation of the polymeric microspheres, being hydrolyzed by an acid or a base, and being hydrolyzed into water-soluble products. In general, compounds having backbone of amide, ester, anhydride and halogen acid are known to be easily hydrolyzed by an acid or a base. Compounds having backbone of anhydride are hydrolyzed to produce water-soluble carboxylic acids, and compounds having backbone of ester are hydrolyzed into carboxylic acid and alcohol. Compounds having backbone of acid halogen are hydrolyzed into carboxylic acid and halogen acid (HF, HCl, Hbr, HI etc). Since compounds having backbone of amide are hydrolyzed into carboxylic acid and amine, if the produced amine is water-soluble, the corresponding amide may be included in the water-insoluble organic solvent of the present invention.

The water-insoluble organic solvent of the present invention may be compounds having backbone of acid halogen, compounds having backbone of anhydride, compounds having backbone of phosphoric anhydride, compounds having backbone of ester, compounds having backbone of carboxylic esters, compounds having backbone of phosphoric esters, compounds having backbone of sulfuric acid esters, compounds having backbone of nitric esters, compounds having backbone of boric acid, compounds having backbone of amide and compounds having backbone of carboxylic amides, preferably, methyl acetate, ethyl acetate, propyl acetate, isopropyl acetate, butyl acetate, methyl formate, ethyl formate, isopropyl formate, propyl formate, butyl formate, methyl dichloroacetate, methyl chloroacetate, ethyl chloroacetate, ethyl dichloroacetate, methyl fluoroacetate, methyl difluoroacetate, ethyl fluoroacetate, ethyl difluoroacetate, maleic anhydride, acetic anhydride, propionic anhydride, phosphoric anhydride, acetamide, propionamide, butylamide and carboxyl amide.

More specifically, it is preferable that the water-insoluble organic solvent is selected from the group consisting of ethyl acetate, methyl acetate, methyl formate, ethyl formate, isopropyl formate, propyl formate, acetic anhydride or propionic anhydride.

The dispersion solvent used in the present invention includes an aqueous dispersion solvent containing an emulsifier or non-aqueous dispersion solvent, and the aqueous dispersion solvent is used for the preparation of an O/W type and W/O/W type emulsion, the non-aqueous dispersion solvent is used for the preparation of an O/O type emulsion. As the aqueous dispersion solvent, an aqueous solution containing hydrophilic emulsifier such as polyvinyl alcohol or polysorbates (for example, polysorbate 20, polysorbate 60, polysorbate 65, polysorbate 80, polysorbate 85) or a co-solvent thereof can be used. As the non-aqueous dispersion solvent, silicone oil, vegetable oil, toluene, or xylene containing lipophilic emulsifier such as glycerin esters of fatty acids or lecithin can be used. The concentration of the emulsifier in the dispersion solvent may be 0.05 to 15% (w/v).

The amount of the water-insoluble organic solvent to be mixed with the dispersion solvent may vary according to the kind of the polymer compound used in the preparation of the polymeric microsphere, the kind of the drug to be loaded, and the kind of the dispersion solvent. Preferably, the water-insoluble organic solvent may be added in an amount less than its water solubility. When it is mixed in an excessively small amount, a polymeric microsphere has a porous surface structure, thereby increasing an initially released amount of a drug. Meanwhile, when it is added in an amount more than its water solubility, it is difficult to remove the organic solvent, thereby increasing the concentration of the remaining organic solvent.

In the step (b), a polymer compound, a drug and a water-insoluble organic solvent are mixed so as to prepare a dispersed phase.

In the present invention, “a dispersed phase” indicates that a polymer compound and a drug are dissolved in and mixed with a water-insoluble organic solvent. The example of the water-insoluble organic solvent in the step (b) is the same as that described in the step (a).

In the step (b), the water-insoluble organic solvent may be preferably the same kind of organic solvent as that used in the step (a). As required, as the water-insoluble organic solvent, a cosolvent obtained by mixing the water-insoluble organic solvent with at least one kind of other organic solvent may be used so that the solubility of a drug to be loaded in the microsphere can be adjusted or the hardening rate of emulsion drops can be controlled as desired.

There is no limitation in the polymer compound used in the preparation method of the present invention, as long as it is a polymer compound known in the art. Preferably, the polymer compound may be selected from the group including polylactic acid, polylactide, polylactic-co-glycolic acid, polylactide-co-glycolide (PLGA), polyphosphazene, polyiminocarbonate, polyphosphoester, polyanhydride, polyorthoester, lactic acid-caprolactone copolymer, polycaprolactone, polyhydroxyvalerate, polyhydroxybutyrate, polyamino acid, lactic acid-amino acid copolymer, and a mixture thereof. More preferably, the polymer compound may be polylactide-co-glycolide (PLGA).

Also, preferably, the polymer compound used in the preparation method of the present invention may be a polymer compound whose end is treated so that it is not subjected to hydrolysis by acid or base. For example, the polymer compound may be PLGA with an esterified end, PCL with an esterified end, or polyanhydride with an esterified end.

The drug used in the present invention may include all of hydrophilic drugs and hydrophobic drugs and it may be used without limitation if it is able to be encapsulated to polymeric microspheres. Examples of the drug include progesterone, haloperidol, thiothixene, olanzapine, clozapine, bromperidol, pimozide, risperidone, ziprasidone, diazepam, ethyl loflazepate, alprazolam, nemonapride, fluoxetine, sertraline, venlafaxine, donepezil, tacrine, galantamine, rivastigmine, selegiline, ropinirole, pergolide, trihexyphenidyl, bromocriptine, benztropine, colchicine, nordazepam, etizolam, bromazepam, clotiazepam, mexazolum, buspirone, goserelin acetate, somatotropin, leuprolide acetate, octreotide, cetrorelix, sandostatin acetate, gonadotropin, fluconazole, itraconazole, mizoribine, cyclosporin, tacrolimus, naloxone, naltrexone, cladribine, chlorambucil, tretinoin, carmustine, anagrelide, doxorubicin, anastrozole, idarubicin, cisplatin, dactinomycin, docetaxel, paclitaxel, raltitrexed, epirubicin, letrozole, mefloquine, primaquine, oxybutynin, tolterodine, allylestrenol, lovostatin, simvastatin, provastatin, atrovastatin, alendronate, salcatonin, raloxifene, oxadrolone, conjugated estrogen, estradiol, estradiol valerate, estradiol benzoate, ethinyl estradiol, etonogestrel, levonorgestrel, tibolone, norethisterone and piroxicam and it also may be macro molecules such as proteins or nucleic acid.

The polymer compound may be used in an amount of 1 to 500 parts by weight, preferably of 1 to 50 parts by weight, with respect to 1 part by weight of a drug.

In the step (c), the dispersed phase in the step (b) is mixed with the dispersion solvent mixed with the water-insoluble organic solvent in the step (a) so as to prepare an O/W (oil-in-water), O/O (oil-in-oil) or W/O/W (water-in oil-in-water) type emulsion.

In the preparation of the O/W (oil-in-water) type emulsion, a polymer compound, a drug and a water-insoluble organic solvent are mixed to prepare a dispersed phase, and the dispersed phase is mixed with a dispersion solvent added with a water-insoluble organic solvent to prepare the O/W (oil-in-water) type emulsion. In the preparation of the O/O (oil in oil) type emulsion, a polymer compound, a drug and an organic solvent are mixed to prepare a dispersed phase, and the dispersed phase is mixed with a dispersion solvent including an organic solvent not to be mixed with the previously used organic solvent to prepare the O/O (oil in oil) type emulsion. In the preparation of the W/O/W (water-in-oil-in-water) type emulsion, an aqueous solution having a drug dissolved therein is emulsified in a water-insoluble organic solvent having a polymer compound dissolved therein to prepare a W/O (water-in-oil) type emulsion, and the prepared emulsion is mixed again with a dispersion solvent added with a water-insoluble organic solvent to prepare a W/O/W (water-in-oil-in-water) type emulsion.

In the step (c), the dispersed phase and the dispersion solvent mixed with the water-insoluble organic solvent are mixed preferably in a volumetric ratio of 1:3 to 100, most preferably of 1:4 to 20.

When the ratio of the dispersion solvent is less than the above mentioned range, emulsion formation is not sufficiently carried out, and when the ratio is greater than the range, a waste solution excessively increased.

In the step (d), the emulsion prepared in the step (c) is added with a base or an acid so as to remove the water-insoluble organic solvent from the emulsion.

In the present invention, the step of adding a base or acid solution so as to remove the water-insoluble organic solvent from the emulsion is preferably carried out by a hydrolysis reaction. The hydrolysis reaction is a reaction where a corresponding material is split into two materials through the addition of water. In the hydrolysis reaction, a compound having an ester structure is hydrolyzed into carboxylic acid and alcohol, a compound having an anhydride structure is hydrolyzed into carboxylic acid, a compound having an amide structure is hydrolyzed into carboxylic acid and amine, and a compound having a halogen acid structure is hydrolyzed into carboxylic acid and halogen acid (HF, HCl, HBr, HI or the like). Through the reaction, the water-insoluble organic solvent diffused (or dissolved) in a small amount in one layer (e.g., a water phase) can be converted into a water soluble organic solvent capable of being completely dissolved in water, and by the converted amount, the water-insoluble organic solvent can be diffused into the water phase. While this process is continuously carried out, the water-insoluble organic solvent is removed from the inside of the emulsion, thereby hardening emulsion drops into microspheres. Thus, it is possible to prepare required drug-containing polymeric microspheres. The above described removal of the water-insoluble organic solvent from the inside of the emulsion indicates not only that the water-insoluble organic solvent is completely or actually (up to an extent where it is not detected) removed, but also that the amount of the water-insoluble organic solvent is reduced compared to that in the initial stage before the introduction of acid or base. Herein, since the emulsion drops are quickly hardened, interaction between emulsion drop particles is inhibited. This allows a required polymeric microsphere to be obtained without agglomeration. Herein, the acid catalyzes the reaction and the base is consumed by the reaction. Once the base or the acid is added, there is no particular problem in the reaction although the amount is less or greater than that of the water-insoluble organic solvent. However, addition of too many moles of acid or base may cause a problem in the stability of the drug and the polymer compound. Thus, it is required to consider a proper amount.

A basic solution may be added in such a manner that a molar ratio of the water-insoluble organic solvent to the basic solution can be preferably in a range of 1:0.1 to 10, more preferably of 1:0.2 to 5, much more preferably of 1:0.3 to 3, and most preferably of 1:0.5 to 1.5.

The temperature of the emulsion in the steps (c) and (d) may vary according to the kinds of the polymer compound, the drug, the water-insoluble organic solvent, and the base or acid, and preferably may be in a range of 0° C. to 35° C.

When the temperature of the emulsion is greater than 35° C., the drug and the polymer compound may be decomposed according to the kinds of the drug, the polymer compound, the base or acid. When the temperature is less than 0° C., the water soluble dispersion solvent is frozen. Thus, emulsion formation cannot be smoothly carried out.

The base may be preferably selected from the group including sodium hydroxide (NaOH), lithium hydroxide (LiOH), potassium hydroxide (KOH), ammonium hydroxide (NH₄OH), copper hydroxide (Cu(OH)₂), and iron hydroxide (Fe(OH)₃), and the acid may be preferably selected from the group including hydrochloric acid (HCl), nitric acid (HNO₃), sulfuric acid (H₂SO₄), acetic acid (CH₃COOH), boric acid (H₃BO₃) and carbonic acid (H₂CO₃).

As described above, according to the present invention, it is possible to conveniently prepare a drug-containing polymeric microsphere within a short time by using a small amount of water, with the generation of a minimized amount of waste solution without a requirement of a conventional solvent-evaporation or solvent-extraction process. Also, according to the method for preparing a polymeric microsphere, according to the present invention, it is possible to prepare a polymeric microsphere with a low concentration of the organic solvent remaining within the polymeric microsphere.

According to other embodiments, the present invention provides methods for preparing a polymeric microsphere, which include the steps of: (a) mixing a water-insoluble organic solvent with a dispersion solvent; (b) mixing a polymer compound, a drug and a water-insoluble organic solvent so as to prepare a dispersed phase; (c) mixing the dispersed phase in the step (b) with the dispersion solvent mixed with the water-insoluble organic solvent in the step (a) so as to prepare an O/W (oil-in-water), O/O (oil-in-oil) or W/O/W (water-in oil-in-water) type emulsion; (d) adding a base or an acid to the emulsion prepared in the step (c) so as to remove the water-insoluble organic solvent from the emulsion; and (e) obtaining the polymeric microsphere prepared in the step (d), whose water-insoluble organic solvent is removed, and re-dispersing the obtained polymeric microsphere in a warmed dispersion solvent.

Hereinafter, a method for preparing a polymeric microsphere, according to the present invention, will be described in detail in respective steps.

The steps (a) to (d) are the same as those described above. In step (e), the polymeric microsphere prepared in the step (d), whose water-insoluble organic solvent is removed, is obtained, and then re-dispersed in a warmed dispersion solvent.

The dispersion solvent used in the re-dispersion of the present invention includes an aqueous dispersion solvent or a non-aqueous dispersion solvent which contains an emulsifying agent. In the preparation of an O/W or W/O/W type emulsion, the aqueous dispersion solvent may be used, and in the preparation of an O/O type emulsion, the non-aqueous re-dispersion solvent may be used. As the aqueous dispersion solvent, an aqueous solution or a cosolvent thereof may be used, which contains a hydrophilic emulsifier, such as polyvinyl alcohol and Polysorbate-based (e.g., polysorbate 20, polysorbate 60, polysorbate 65, polysorbate 80, polysorbate 85) emulsifier. As the non-aqueous dispersion solvent, silicon oil, vegetable oil, toluene, or xylene may be used, which contain a hydrophobic emulsifier, such as Glycerin Esters of Fatty Acids, or lecithin. The concentration of the emulsifier contained in the dispersion solvent may be in a range of 0.05 to 15% (w/v).

The temperature of the warmed dispersion solvent may vary according to the kinds and amounts of the drug, the water-insoluble organic solvent, and the polymer compound, and may be preferably in a range of 20° C. to 80° C., more preferably of 30° C. to 50° C., and most preferably of 30° C. to 40° C. When the temperature of the dispersion solvent is less than 20° C., the amount of remaining organic solvent may be increased. Then the temperature is greater than 80° C., the polymeric microsphere may be deformed.

In the polymeric microsphere re-dispersed in the warmed dispersion solvent, the concentration of the organic solvent within the microsphere is further reduced.

As described above, in the inventive method, it is possible to conveniently prepare a drug-containing polymeric microsphere within a short time by using a small amount of water, with the generation of a minimized amount of waste solution without a requirement of a conventional solvent-evaporation or solvent-extraction process. Also, it is possible to reduce the concentration of the organic solvent remaining within the prepared polymeric microsphere.

Meanwhile, the present invention provides the polymeric microsphere prepared by the above described inventive method.

The polymeric microsphere prepared by the inventive method has a low concentration of remaining organic solvent, in which the releasing speed of a drug within a body can be controlled.

The polymeric microsphere prepared by the inventive method has an average diameter in a range of 0.1 to 1000 μm, and preferably of 10 to 100 μm, and may contain a drug in various weights as required.

Also, when the inventive polymeric microsphere is used, it is possible to effectively deliver the drug contained in the polymeric microsphere. Thus, the present invention provides a drug delivery composition including, as an active ingredient, the polymeric microsphere prepared by the inventive preparation method.

The inventive drug delivery composition has different target diseases according to its contained drugs, which may be easily understood by a person skilled in the art.

In Example 1, a polymeric microsphere was prepared while the concentration of a water-insoluble organic solvent mixed with a dispersion solvent is varied. Then, according to such a concentration change, the concentration of remaining solvent was measured. As a result, it was found that as long as the dispersion solvent is previously added with the organic solvent, the concentration of the remaining organic solvent is reduced up to a maximum of about 25%, irrespective of drug inclusion.

In Example 2-1, concentrations of a remaining solvent according to the temperature of a dispersion solvent in a re-dispersion step were compared. A polymeric microsphere preparation process was divided into two steps. In the first step, a base or acid solution is added and the reaction is completed, and in the second step, after the completion of the reaction, a microsphere is filtered and separated, and then is re-dispersed in PVA solution, followed by stirring. In the preparation of a polymeric microsphere containing 60 mg of olanzapine, one group of polymeric microspheres was prepared while the temperatures of the first and second steps were warmed and maintained up to 33° C. On the other hand, the other group of polymeric microspheres was prepared while the temperature of only one of the first and second steps was warmed and maintained up to 33° C. Then, the remaining solvents were measured. As a result, it was found that irrespective of the warmed temperature in the first step, the warmed temperature in the second step significantly reduced the concentration of the solvent remaining in the polymeric microsphere, compared to the case where in the second step, the temperature is not warmed.

In Example 2-2, the same result as the above described result was obtained when another kind of PLGA was used to repeatedly carry out the test.

In Example 2-3, a polymeric microsphere was prepared by increasing the content of a polymer and the amount of a drug, and the amount of remaining solvent was measured. As a result, it was found that when the temperature in the re-dispersion step (second step) is warmed, it is possible to maintain a low amount of the remaining solvent.

In Example 3, concentrations of a remaining solvent according to the concentrations of a water-insoluble organic solvent mixed with a dispersion solvent were compared. A polymer compound was dissolved in ethyl formate so as to provide a dispersed phase. The dispersed phase was emulsified in 20 ml of 0.5% PVA aqueous solution so as to provide an emulsion, in which the PVA aqueous solution was previously added with 0, 1, 2 or 4 ml of ethyl formate. Then, base was added to remove the water-insoluble organic solvent, and then the polymeric microsphere was prepared. Then, the amount of the remaining solvent was measured, and the polymeric microsphere was photographed.

As a result, it was found that as the concentration of an organic solvent previously added to a dispersion solvent is increased, the concentration of the solvent remaining in the polymeric microsphere is decreased. Furthermore, it was found that when an organic solvent is previously added to a dispersion solvent, it is possible to stably prepare a spherical polymeric microsphere.

In Example 4, without a step of previously mixing a water-insoluble organic solvent with a dispersion solvent, polymeric microspheres were prepared by varying a ratio of a dispersed phase to the dispersion solvent. Then, according to a change of a ratio of a dispersed phase to a dispersion solvent, a change of the concentration of a remaining solvent was measured. A polymeric microsphere was prepared while the ratio of the dispersed phase to the dispersion solvent was adjusted to 1:10, 1:6 or 1:4. Then, the amount of remaining solvent was measured.

As a result, when the ratio of the dispersed phase and the dispersion solvent is 1:4 or 1:6, the amount of a remaining solvent (EA or EF) is reduced, compared to that in 1:10. However, it was found that the amount of remaining ethanol is increased. Thus, it was determined that it is possible to adjust the concentration of solvent remaining in the polymeric microsphere by adjusting the ratio of the dispersed phase to the dispersion solvent. Further, it was found that it does not matter which one of the dispersed phase and the dispersion solvent in the ratio of the dispersed phase and the dispersion solvent was adjusted.

In Example 5-1, by varying the ratio of a dispersed phase to a dispersion solvent, and the amount of an organic solvent previously mixed with the dispersion solvent, and maintaining the temperature in the re-dispersion step at 40° C., polymeric microspheres were prepared. The change in the characteristics was measured. The dispersed phase was immobilized by 4 ml of ethyl formate including 250 mg of polymer compound dissolved therein. Then, the dispersion solvent was used in an amount of 20, 30, or 40 ml, and the amount of the organic solvent previously added to the dispersion solvent was increased from 0% to 10% with respect to the dispersion solvent so as to provide a polymeric microsphere

As a result, when the amount of the remaining solvent was measured, compared to the case where no ethyl acetate was added to the dispersion solvent, the addition of ethyl acetate reduced the concentration of the remaining solvent. Especially, the amount of the remaining solvent was highly increased when a ratio of the dispersed phase to the dispersion solvent was 1:10 (40 ml of dispersion solvent PVA) and the dispersion solvent was not previously added with EF.

In Example 5-2, the yield of the prepared polymeric microsphere was measured. As a result, it was determined that there is no problem in the yield of the polymeric microsphere.

In Example 5-3, a molecular weight change of a polymer compound used in the polymeric microsphere preparation, before/after the preparation, was measured. As a result, it was found that there was no significant difference in the molecular weights between the polymer compounds before/after the preparation.

In Example 6, a molecular weight change of a polymer compound before/after the preparation of a drug-containing polymeric microsphere was measured. The polymeric microsphere was prepared by varying the kind of a polymer compound, the kind and the content of a drug, and the temperature in first and second steps. Then, the molecular weight of the polymer compound before/after the preparation was measured.

As a result, it was found that polymer compounds of all the polymeric microspheres show no change in their molecular weights. Thus, it was determined that in the inventive preparation method, a molecular weight of a polymer compound can be well maintained irrespective of conditions such as the kind of a drug, or the reaction temperature.

In Example 7-2, an olanzapine-containing polymeric microsphere was prepared according to the inventive method.

For the prepared olanzapine-containing polymeric microsphere, the amount of remaining solvent, the loading efficiency of olanzapine, and the yield of the polymeric microsphere were measured. As a result, it was found that the low concentration of the remaining solvent was maintained, and almost all the loading efficiencies and the yields showed a high value of 70% or more.

In Example 8-2, a polymeric microsphere was prepared by using another water-insoluble organic solvent rather than the water-insoluble organic solvent used in Examples 1 to 7. By using ethyl acetate as the water-insoluble organic solvent, the polymeric microsphere was prepared. Then, the amount of remaining solvent, the loading efficiency of olanzapine, and the yield of the polymeric microsphere were measured. As a result, it was found that the small amount of the remaining solvent was maintained, and almost all the loading efficiencies and the yields showed high values of 85% or more and 70% or more, respectively.

In Example 9, an olanzapine-containing polymeric microsphere was prepared, and its dispersibility and injectability were tested. Then, it was injected into a rat. By measuring the concentration of olanzapine in blood, a pharmacokinetic test was carried out.

As a result, it was determined that the inventive composition including an olanzapine-containing polymeric microsphere is good in dispersibility and injectability, and thus can be used as a parenteral injection. Furthermore, it was found that when the composition was injected into a rat, olanzapine in blood was continuously released for a maximum of 80 days.

In Example 10-2, a polymeric microsphere containing anastrozole instead of olanzapine was prepared.

The yield and the loading efficiency of the prepared polymeric microsphere were almost 80% or more, which is appropriate irrespective of the kind of PLGA. Also, the average particle size was found to have an appropriate value ranging from 30 to 60 um. The remaining solvent was included at a very low concentration, which was found to be appropriate irrespective of the kind of a test sample. Also, through the photograph by the electron microscope, it can be found that the polymeric microsphere was generally well prepared in a smooth spherical shape irrespective of the kind of used PLGA. Especially, it was found that even when the drug content was increased up to 40%, the drug was well loaded and no crystallization of the drug was observed from the surface.

In Example 10-3, through GPC analysis, the molecular weight change of a prepared polymeric microsphere was measured. As a result, it was found that most polymers show no change in their molecular weights. In order to analyze a related compound within the prepared polymeric microsphere, the prepared anastrozole-containing polymeric microsphere was subjected to ultra performance liquid chromatography (UPLC). As a result, it was found that the average amount of the related compound in the inventive polymeric microsphere was very low (0.17%).

Also, when a stability test was carried out for 2 months under a severe condition, the content of the related compound was less than 0.23%, and a change of the drug content was less than 10%. Thus, it was found that the polymeric microsphere prepared by the inventive preparation method has a high stability.

In Example 10-4, the persistence of a prepared polymeric microsphere was tested by introducing the inventive polymeric microsphere in a dialysis membrane, and changing a buffer at a predetermined time interval.

As a result, it was determined that after the drug release, polymeric microspheres of all formulations hardly have remaining drugs. Also, according to the kinds of the polymer compounds used in the preparation, as the ratio of lactide was increased, the drug was slowly released. Meanwhile, it was determined that as drug content was increased, the release rate was increased. Thus, it was determined that it is possible to adjust the release rate by adjusting the kind of the polymer compound used in the polymeric microsphere preparation, and the amount of the loaded drug.

In Example 10-5, the persistence of a prepared polymeric microsphere was measured through an animal test.

The prepared anastrozole-containing polymeric microsphere was administered in an amount of 20 mg/kg to a rat through intramuscular injection, and then the concentration of anastrozole in blood was measured.

As a result, it was found that when the polymeric microsphere prepared by the inventive method was injected, a drug can be continuously released for a maximum of 120 days according to the kind of the prepared polymeric microsphere.

### EXAMPLES

Hereinafter, the present invention will be described in detail with reference to the Examples. However, Examples as described below are only for illustrative purposes and are not intended to limit the scope of the invention.

### Example 1

### Comparison of Concentrations of a Remaining Solvent According to Concentrations of a Water-Insoluble Organic Solvent Mixed with a Dispersion Solvent

<1-1> Measurement of a Remaining Solvent in the Preparation of a Polymeric Microsphere According to a Conventional Method

0.25 g of 7525 2.5E PLGA polymer was dissolved in 4 ml of ethyl formate (EF), and then emulsified in 40 ml of 0.5% polyvinyl alcohol (PVA) so as to provide an emulsion. Then, 3.4 ml of 28% NH3 solution was added to the emulsion, followed by a reaction for 30 minutes. The resultant product was added with distilled water, and filtered. A microsphere was separated, and then re-dispersed in 80 ml of 0.1% PVA, followed by stirring. The information on 7525 2.5E PLGA polymer used in this example is noted in Table 1.

TABLE 1
Information on 7525 2.5E PLGA polymer

| | | Lactide: | Inherent |
|---|---|---|---|
| Name and trademark | manufacturer | Glycolide | viscosity |
| 7525DLG2.5E | SurModics | 75:25 | 0.25 dL/g |
| (LAKESHORE- | Pharmaceuticals | | |
| BIOMTERIALS) | Co. (US) | | |

An analysis on a remaining solvent of a microsphere was carried out by gas chromatography (GC) as described below. The GC device was GC-2010 (shimadzu, Japan), and the column was ZB-624 (phenomenex, US). The temperature of SPL was maintained at 200° C., and the sample had a split ratio of 15. As the carrier gas, high purity nitrogen gas was used. The pressure was maintained at 54.3 kPa (flow rate: 1.3 ml/min) for 2 minutes, and maintained at a rate of −50° C. at 40 kPa for 3 minutes. Then, the pressure was raised up to 100 kPa at a rate of 80, and maintained for 2 minutes. The temperature of the column was maintained at 80° C. for 5.1 minutes, and then raised up to 180° C. at a rate of 200° C. per min, and maintained for 2 minutes. As a detector, a flame ionization detector (FID) was used, and the temperature was 220° C. About 50 mg of a polymeric microsphere sample was collected and the weight was exactly measured. Then, the sample was completely dissolved in 2 ml of tetrahydrofuran. The resultant solution was diluted up to 4 times by using pentanol, and the precipitated polymer was filtered, and injected into the GC.

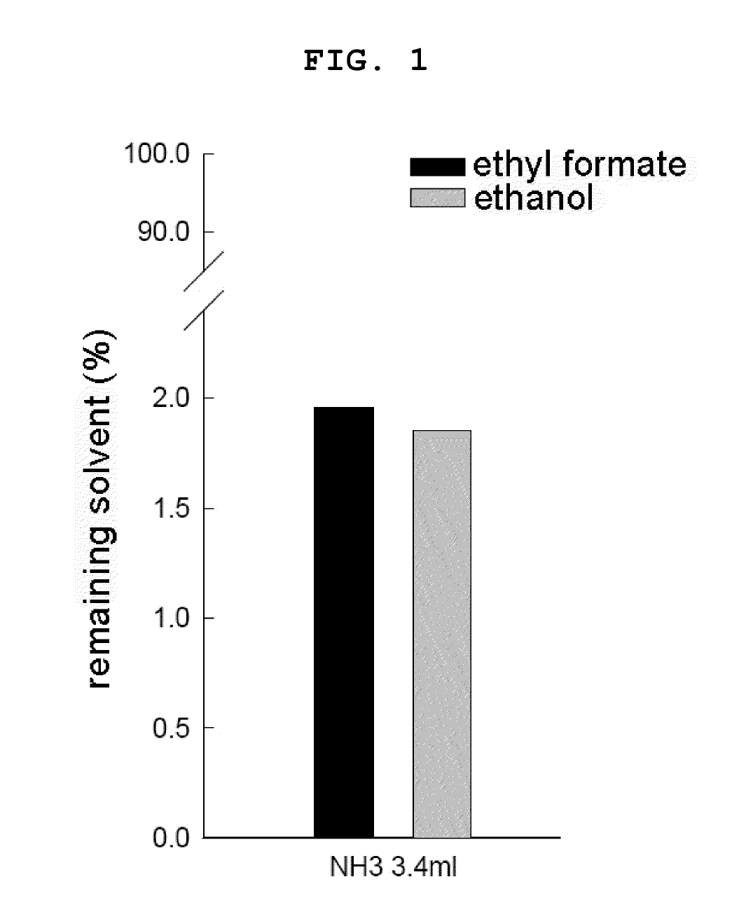

As a result, as shown in FIG. 1, it was found that the remaining solvent was 1.5% or more.

<1-2> Concentrations of a Remaining Solvent According to Concentrations of a Water-Insoluble Organic Solvent Mixed with a Dispersion Solvent

The concentration of an organic solvent remaining in a polymeric microsphere was measured according to whether or not a dispersion solvent is previously mixed with a water-insoluble organic solvent

A polymeric microsphere was prepared according to polymeric microsphere preparation conditions noted in Table 2 below.

TABLE 2
polymeric microsphere preparation conditions

| | Amount of | | | | |
|---|---|---|---|---|---|
| | organic | | | | |
| | solvent (EF) | | | | |
| | mixed with | Amount of | | Amount of | Amount of |
| | dispersion | drug | Amount of | remaining | remaining |
| No | solvent | (olanzapine) | NaOH | EF (%) | ethanol (%) |
| A | 0 ml | 0 mg | 5 ml | 0.62 | 0.48 |
| B | 0.5 ml | 0 mg | 5.6 ml | 0.34 | 0.52 |
| C | 1.0 ml | 0 mg | 6.2 ml | 0.16 | 0.46 |
| D | 0 ml | 60 mg | 5 ml | 0.55 | 0.94 |
| E | 0.5 ml | 60 mg | 5.6 ml | 0.31 | 1.03 |
| F | 1.0 ml | 60 mg | 6.2 ml | 0.21 | 0.87 |

First, 0.35 g of 7525 2.5E polylactide-co-glycolide (PLGA) polymer and olanzapine according to Table 1 were dissolved in 4 ml of ethyl formate (EF), and emulsified in 20 ml of 0.5% Poly(vinyl Alcohol) (PVA) previously added with EF organic solvent according to Table 1 so as to provide an emulsion. 10 N NaOH was added to the emulsion, followed by a reaction for 30 minutes. The resultant product was added with distilled water, and filtered. A microsphere was separated, and then re-dispersed in 80 ml of 0.1% PVA, followed by stirring.

As a result, as noted in Table 2, it was found that when the dispersion solvent is added with an organic solvent, the concentration of the remaining organic solvent is reduced.

### Example 2

### Comparison of Concentrations of a Remaining Solvent According to the Temperature of a Dispersion Solvent in a Re-Dispersion Step

In the preparation of a polymeric microsphere, it was expected that the reaction of acid or base for removing a water-insoluble organic solvent changes the temperature of an aqueous phase, thereby having an effect on the amount of a remaining solvent. Accordingly, the polymeric microsphere preparation process was divided into two steps. In the first step, a base or acid solution is added and the reaction is completed, and in the second step, after the completion of the reaction, a microsphere is filtered and separated, and then is re-dispersed in PVA solution, followed by stirring. In this example, it was determined which step has a temperature as an important role for reducing the remaining solvent.

<2-1> Comparison of Concentrations of a Remaining Solvent According to the Temperature of a Dispersion Solvent in a Re-Dispersion Step in the Polymeric Microsphere Preparation Using 7525 2.5E PLGA

The polymeric microsphere preparation process is carried out as described below. 60 mg of olanzapine and 250 mg of 7525 2.5E PLGA were dissolved in 4 ml of ethyl formate. The dispersed phase was emulsified in 20 ml of 0.5% PVA aqueous solution having 1 ml of ethyl formate previously dissolved therein so as to provide an emulsion. Then, 6.2 ml of 10N NaOH was added to the emulsion, followed by a reaction for 30 minutes so as to provide a polymeric microsphere. The polymeric microsphere was separated, and then re-dispersed in 80 ml of 0.1% PVA, followed by stirring. Then, the polymeric microsphere was separated and vacuum-dried.

In the process, the first and second steps were performed, respectively, under the temperature conditions noted in Table 3 below, and the remaining solvents were measured according to the method as described in Example 1.

TABLE 3
Concentrations of a remaining solvent according
to the preparation conditions in the polymeric
microsphere preparation using 7525 2.5E PLGA

| | Amount of | Amount of |
|---|---|---|
| | remaining | remaining |
| Preparation condition | EF(%) | EtOH(%) |
| Step 1: heated up to 33° C. | 0.12 | 0.33 |
| Step 2: heated up to 33° C. | | |
| Step 1: not heated | 0.09 | 0.36 |
| Step 2: heated up to 33° C. | | |
| Step 1: heated up to 33° C. | 0.10 | 0.68 |
| Step 2: not heated | | |

As a result, as noted in Table 3, when in the second step, heating is not carried out, the amount of remaining ethanol within a polymeric microsphere is greater than 0.5%. On the other hand, when the aqueous phase is maintained at 33° C. in the second step, the amount of remaining ethanol was reduced to less than 0.5%. Meanwhile, as long as the temperature of the aqueous phase in the second step was maintained at 33° C., there is no significant difference in the concentration of the remaining solvent between the cases where the temperature in the first step was maintained at 33° C., and the temperature was not maintained.

Accordingly, it was determined that the control of the temperature of an aqueous phase in the step performs an important role of reducing the remaining solvent.

<2-2> Comparison of Concentrations of a Remaining Solvent According to the Temperature of a Dispersion Solvent in a Re-Dispersion Step in the Polymeric Microsphere Preparation Using 6535 4.5A PLGA

In general, as the molecular weight of PLGA is increased, the amount of the organic solvent remaining in the polymeric microsphere is increased. Accordingly, it was tested whether the problem of a remaining organic solvent can be overcome in a case where 6535 4.5A PLGA having a high molecular weight is used in the inventive method.

The information on the PLGA polymer used in the Example is noted in Table 4.

TABLE 4
Information on 6535 4.5A PLGA polymer

| | | Lactide: | Inherent |
|---|---|---|---|
| Name and trademark | manufacturer | Glycolide | viscosity |
| 6535DLG4.5A | SurModics | 64:36 | 0.49 dL/g |
| (LAKESHORE | Pharmaceuticals | | |
| BIOMTERIALS) | Co. (US) | | |

The process of preparing a polymeric microsphere is the same as that described in Example 2-1 except that as a polymer, 6535 4.5A PLGA was used, instead of 7525 2.5E PLGA.

In the process, the first and second steps were carried out, respectively, under the temperature conditions as noted in Table 5 below, and then the remaining solvent was measured in the same manner as that described in Example 1.

Also, the state of the prepared polymeric microsphere was observed by an electron microscope (SEM, Hitachi S-3000N, Japan, HV 20 kv, Working Distance 15 mm, Beam current 40).

TABLE 5
Concentrations of a remaining solvent according
to the preparation conditions in the polymeric
microsphere preparation using 6535 4.5A PLGA

| | Amount of | Amount of |
|---|---|---|
| | remaining | remaining |
| Preparation condition | EF (%) | EtOH (%) |
| Step 1: heated up to 33° C. | 0.08 | 0.55 |
| Step 2: heated up to 33° C. | | |
| Step 1: not heated | 0.26 | 1.12 |
| Step 2: not heated | | |

As a result, as noted in Table 5, even in a case where 6535 4.5A was used, it was found that when the temperature of the aqueous phase in the second step was maintained at 33° C., the amounts of remaining ethyl formate and ethanol were significantly reduced.

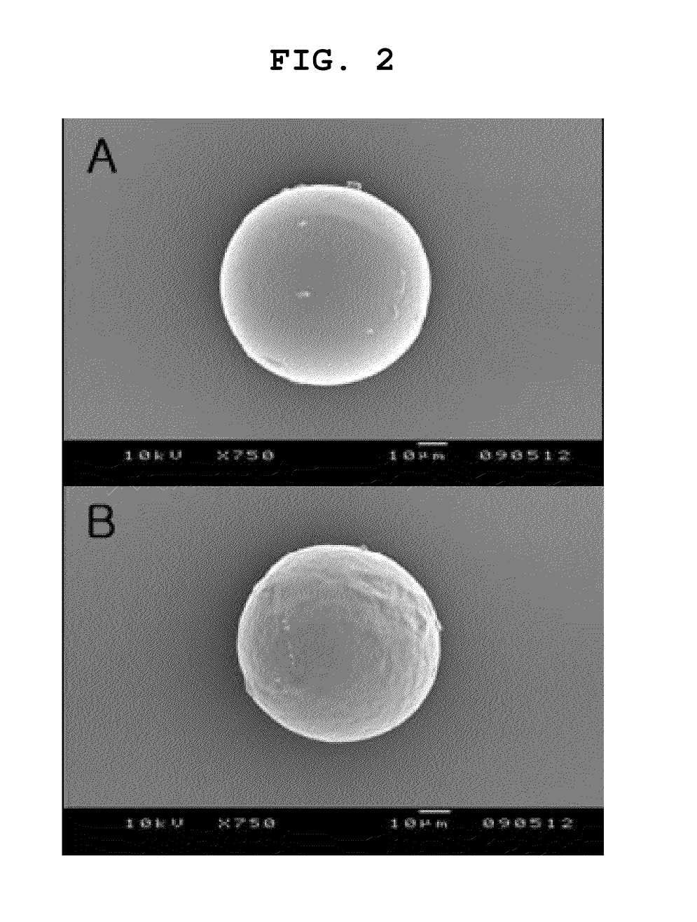

Also, when the polymeric microsphere was photographed by an electron microscope, it was found that the polymeric microsphere having 60 mg of olanzapine sufficiently loaded therein was prepared (see FIG. 2).

<2-3> Comparison of Concentrations of a Remaining Solvent According to the Temperatures of a Dispersion Solvent in the Re-Dispersion Step in a Case where the Use Amount of a Polymer is Increased

It was tested whether the concentration of a solvent remaining in a polymeric microsphere can be reduced according to a temperature change of a dispersion solvent even in a case where the concentration of PLGA in the dispersed phase is increased.

60 mg or 80 mg of olanzapine and 400 mg of 6535 4.5A PLGA were dissolved in 4 ml of ethyl formate so as to provide a dispersed phase. Then, a polymeric microsphere was prepared in the same manner as that described in Example 2-1, and the concentration of the remaining solvent was measured in the same manner as that described in Example 1.

In the process, the temperature in the second step is noted in Table 6.

TABLE 6
Concentrations of a remaining solvent according
to the preparation conditions in the polymeric
microsphere preparation using 6535 4.5A PLGA

| | | | Amount of | Amount of |
|---|---|---|---|---|
| | Temperature (° C.) | Amount of | remaining ethyl | remaining |
| Batch | in the second step | drug (mg) | formate % | ethanol % |
| 1 | 33 | 60 | 0.91 | 0.13 |
| 2 | 33 | 80 | 1.43 | 0.1 or less |
| 3 | 40 | 60 | 0.1 or less | 0.1 or less |
| 4 | 40 | 80 | 0.1 or less | 0.1 or less |
| 5 | 50 | 60 | 0.1 or less | 0.1 or less |
| 6 | 50 | 80 | 0.1 or less | 0.1 or less |

As a result, as noted in Table 6, it was determined that it is possible to maintain a low concentration of a remaining solvent when the temperature in the second step was maintained at 40° C. or more even in a case where the amount of PLGA was increased up to 400 mg.

Also, the state of polymeric microspheres in batches 3 and 4 noted in Table 6 was photographed by an electron microscope (SEM, Hitachi S-3000N, Japan, HV 20 kv, Working Distance 15 mm, Beam current 40).

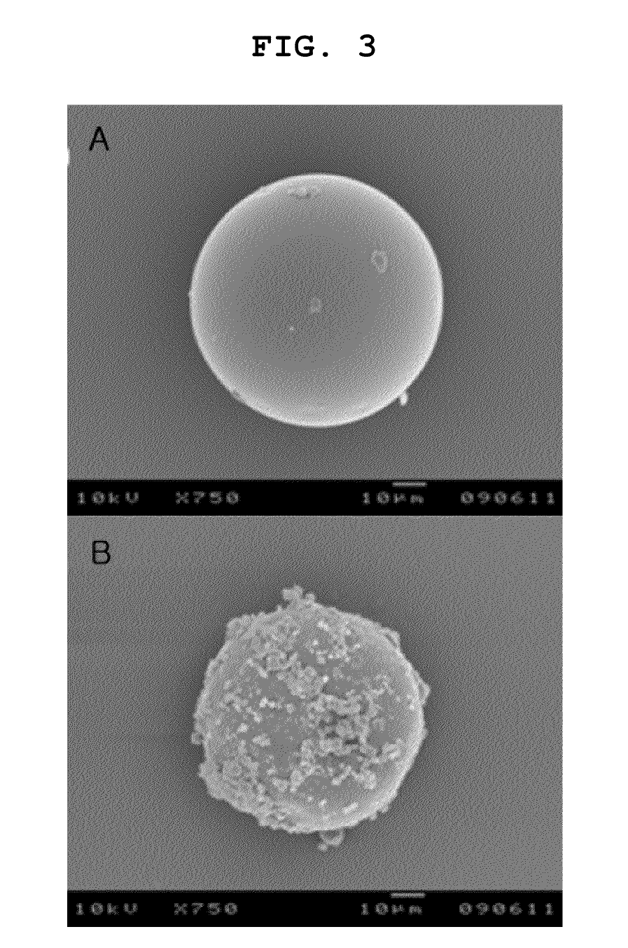

As a result, it was found that a polymeric microsphere having olanzapine loaded therein was prepared (see FIG. 3).

### Example 3

### Comparison 1 of Concentrations of a Remaining Solvent According to the Concentrations of a Water-Insoluble Organic Solvent Mixed with a Dispersion Solvent

In Example 1, it was found that when an organic solvent is previously added to a dispersion solvent, the concentration of the remaining organic solvent is reduced. Accordingly, while the temperature of a dispersion solvent in a re-dispersion step is maintained at a high temperature, the amounts of a remaining solvent were measured according to the varying amounts of a water-insoluble organic solvent mixed with a dispersion solvent.

500 mg of 4.5A PLGA (Lactide:Glycolide=85:15, 0.45 dL/g, SurModics Pharmaceuticals Co., US) was dissolved in 4 ml of ethyl formate so as to provide a dispersed phase. The dispersed phase was emulsified in 20 ml of 0.5% PVA aqueous solution so as to provide an emulsion, in which the PVA aqueous solution was previously added with 0, 1, 2 or 4 ml of ethyl formate. Then, 6.2 ml of 10N NaOH was added to the emulsion, followed by a reaction for 30 minutes, so as to provide a polymeric microsphere. The polymeric microsphere was separated, and then re-dispersed in 80 ml of 0.1% PVA at 40° C., followed by stirring. Then, the polymeric microsphere was separated and vacuum-dried.

After the preparation of the polymeric microsphere, the remaining solvent was measured in the same manner as that described in Example 1, and the prepared polymeric microsphere was photographed in the same manner as that described in Example 2-2.

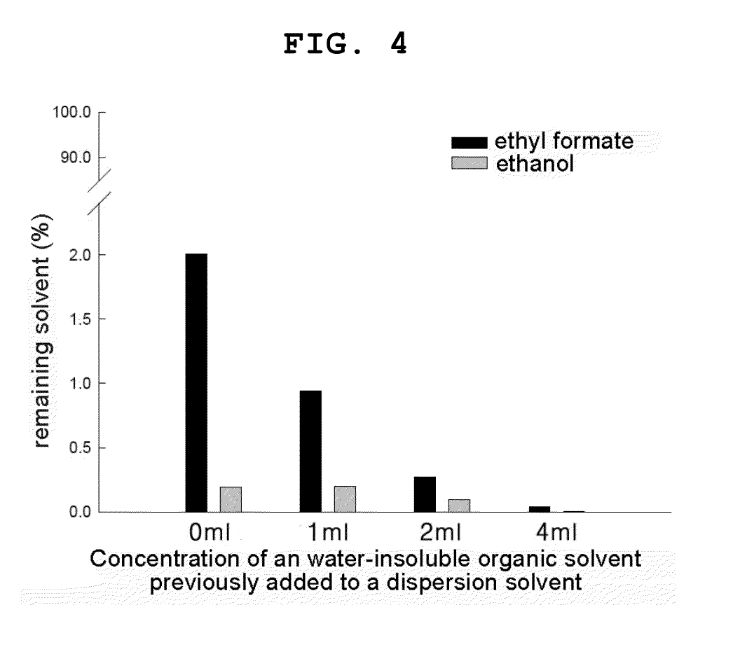

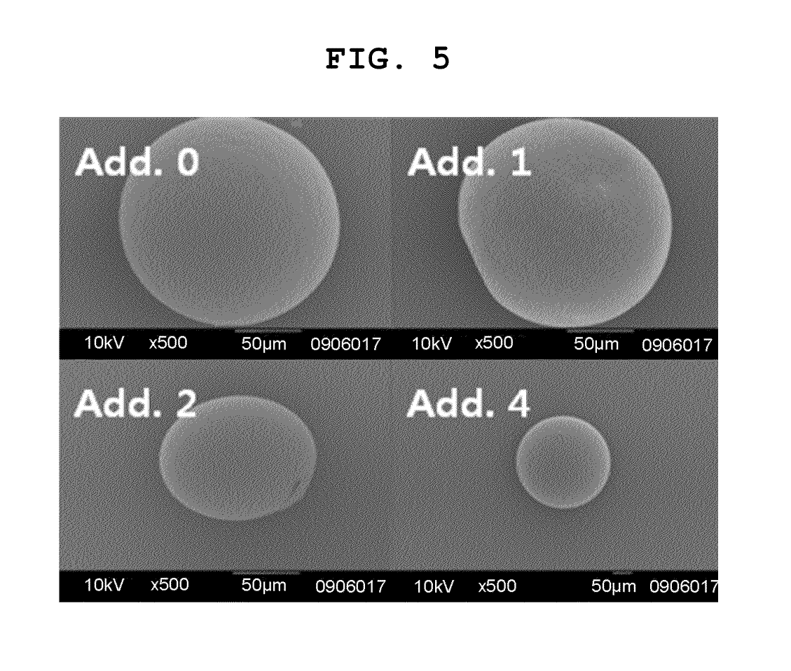

As a result, as shown in FIG. 4, it was found that as the concentration of an organic solvent previously added to a dispersion solvent is increased, the concentration of a solvent remaining in a polymeric microsphere is reduced. Also, as shown in FIG. 5, although an organic solvent is previously added to a dispersion solvent, it is possible to stably prepare a spherical polymeric microsphere. Furthermore, it was found that as the concentration of the organic solvent previously added to the dispersion solvent is increased, the size of the prepared polymeric microsphere is reduced.

### Example 4

### Comparison of Concentrations of a Remaining Solvent According to a Ratio of a Dispersed Phase to a Dispersion Solvent

<4-1> Preparation of a Polymeric Microsphere According to a Change of a Ratio of a Dispersed Phase to a Dispersion Solvent

According to a change of a ratio of a dispersed phase to a dispersion solvent, in order to determine a change of the amount of a remaining solvent, a polymeric microsphere was prepared while the ratio of the dispersed phase (O) to the dispersion solvent (W) is adjusted to 1:10, 1:6 or 1:4 as noted in Table 7. The ratio of the dispersed phase to the dispersion solvent was adjusted by adjusting the amount of the dispersed phase or the amount of the dispersion solvent. Also, as an organic solvent constituting the dispersed phase, ethyl acetate or ethyl formate was used, and as a solvent decomposition reagent, NaOH or ammonia was used.

TABLE 7
A ratio of O/W used in the test and
a solvent decomposition reagent

| | | | | Solvent |
|---|---|---|---|---|
| | | Dispersed | Dispersion | decomposition |
| | O/W ratio | phase (ml) | solvent (ml) | reagent |
| EA-1 | 1:4 | 40 | 160 | NaOH |
| EA-2 | 1:6 | 27 | 160 | NaOH |
| EA-3 | 1:10 | 16 | 160 | NaOH |
| EA-4 | 1:6 | 27 | 160 | NaOH |
| EA-5 | 1:4 | 16 | 64 | NaOH |
| EA-6 | 1:6 | 16 | 96 | NaOH |
| EF-1 | 1:4 | 42.5 | 170 | NH3 |
| EF-2 | 1:6 | 28.3 | 24 | NH3 |
| EF-3 | 1:10 | 17 | 170 | NH3 |
| EF-4 | 1:4 | 17 | 68 | NH3 |
| EF-5 | 1:6 | 17 | 102 | NH3 |
| EF-6 | 1:4 | 40 | 160 | NaOH |
| EF-7 | 1:6 | 27 | 160 | NaOH |
| EF-8 | 1:10 | 16 | 160 | NaOH |
| EF-9 | 1:4 | 16 | 64 | NaOH |
| EF-10 | 1:6 | 16 | 96 | NaOH |

A polymer compound blend 1 g (4A:2A=6:4) and olanzapine 0.333 g were introduced, and an equivalent amount of ethyl acetate or ethyl formate was added thereto. The mixture was completely dissolved, and an equivalent amount of 0.5% PVA was added thereto, followed by stirring at room temperature. An equivalent amount of 10M NaOH or ammonia solution was added thereto, followed by stirring for 30 minutes so as to provide a polymeric microsphere. The prepared polymeric microsphere was filtered and collected, and re-dispersed in 0.1% PVA, followed by stirring. Then, the resultant product was washed with distilled water and freeze-dried.

<4-2> Measurement of the Yield of a Prepared Polymeric Microsphere, and the Remaining Solvent

The remaining solvent of the polymeric microsphere prepared from Example 4-1 was measured in the same manner as that described in Example 1.

The collected polymeric microsphere was placed on a plate (whose weight was previously measured), and then its weight after vacuum-drying was measured. Then, the yield of the polymeric microsphere was calculated by the equation below.

Yield(%)=(weight of collected polymeric microsphere)/(sum of weights of polymer and drug used in the preparation)×100

TABLE 8
The yield of a prepared polymeric microsphere,
and the amount of a remaining solvent

| | Amount of remaining | | |
|---|---|---|---|
| | solvent (%) | | yield |
| | EA | EtOH | (%) |
| EA-1 | 0.023 | 1.273 | 62.5 |
| EA-2 | 0.058 | 1.249 | 38.9 |
| EA-3 | 1.289 | 0.180 | 79.8 |
| EA-4 | 0.113 | 0.997 | 53.9 |
| EA-5 | 0.057 | 0.506 | 54.5 |
| EA-6 | 0.133 | 0.366 | 68.6 |
| EF-1 | 0.249 | 0.199 | 66.8 |
| EF-2 | 0.048 | 0.027 | 26.3 |
| EF-3 | 1.486 | 0.136 | 92.6 |
| EF-4 | 0.530 | 0.070 | 77.9 |
| EF-5 | 0.210 | N.D. | 62.0 |
| EF-6 | N.D. | 1.281 | 43.5 |
| EF-7 | 0.020 | 0.192 | 44.3 |
| EF-8 | 0.365 | N.D. | 71.0 |
| EF-9 | 0.227 | 0.714 | 52.2 |
| EF-10 | 0.739 | 0.562 | 59.5 |

As a result, as noted in Table 8, when the ratio of the dispersed phase and the dispersion solvent is 1:4 or 1:6, the amount of a remaining solvent (EA or EF) is reduced, compared to that in 1:10. However, it was found that the amount of remaining ethanol is increased. Thus, it was found that it doesn t matter which one of the dispersed phase and the dispersion solvent in the ratio of the dispersed phase and the dispersion solvent was adjusted.

### Example 5

### The Change of a Polymeric Microsphere According to the Ratio of a Dispersed Phase to a Dispersion Solvent, the Concentration of a Previously Added Organic Solvent, and a Temperature Change in a Re-Dispersion Step

<5-1> Polymeric Microsphere Preparation and Measurement of a Remaining Solvent

The concentration of a remaining solvent was measured according to the concentration change of a water-insoluble organic solvent mixed with a dispersion solvent, the temperature change of a dispersion solvent in the re-dispersion step (in the second step), and the change of a ratio of a dispersed phase and the dispersion solvent in Examples 1 to 4.

The polymeric microsphere was prepared in consideration of these characteristics, and the concentration of the remaining solvent was measured.

As a dispersed phase, a solution including 250 mg of 4.5A PLGA dissolved in 4 ml of ethyl formate was used. As a dispersion solvent, 20, 30, or 40 ml of 0.5% PVA was used, and a water-insoluble organic solvent (ethyl formate) was previously added to the dispersion solvent in an amount ranging from 0 to 4 ml according to the amount of the dispersion solvent. For the decomposition of the solvent, 10N NaOH was used, which was added in consideration of a total amount of added ethyl formate in a 1:1 equivalent amount. In the preparation of the polymeric microsphere, the first step for adding a base or acid solution and carrying out the reaction was carried out at room temperature, and after the reaction, the second step for re-dispersing the polymeric microsphere filtered and separated in a PVA solution and stirring the mixture was carried out at 40° C.

The analysis of the remaining solvent of the prepared polymeric microsphere was measured in the same manner as that described in Example 1. The preparation process and the remaining solvent analysis were repeatedly carried out twice, and all of the measured values were noted.

TABLE 9
The amount of a remaining solvent of a
prepared polymeric microsphere

| | | Amount | | | | |
|---|---|---|---|---|---|---|
| | | of EF | | | | |
| | | previously | | | | |
| | Amount | added | | | | |
| | of | to | | | | |
| | dispersion | dispersion | Remaining | | Remaining | |
| | solvent | solvent | EF (%) | | ethanol (%) | |
| | (ml) | (ml) | #1 | #2 | #1 | #2 |
| First | 20 | 0 | 0.175 | 0.113 | 0.063 | 0.080 |
| step at | | 1 | 0.053 | 0.015 | 0.044 | 0.049 |
| room | | 2 | 0.036 | 0.006 | 0.044 | 0.056 |
| temperature | 30 | 0 | 0.285 | 0.291 | 0.038 | 0.052 |
| (RT) | | 1 | 0.156 | 0.119 | 0.056 | 0.052 |
| | | 2 | 0.059 | 0.028 | 0.037 | 0.032 |
| | | 3 | 0.042 | 0.020 | 0.078 | 0.041 |
| | 40 | 0 | 0.932 | 1.204 | 0.058 | 0.105 |
| | | 1 | 0.231 | 0.225 | 0.038 | 0.053 |
| | | 2 | 0.164 | 0.079 | 0.040 | 0.043 |
| | | 3 | 0.111 | 0.061 | 0.055 | 0.042 |
| | | 4 | 0.040 | 0.038 | 0.023 | 0.112 |

As a result, as noted in Table 9, compared to the case where no ethyl formate was added to the dispersion solvent, the addition of ethyl formate reduced the concentration of the remaining solvent. Especially, the amount of the remaining solvent was increased when a ratio of the dispersed phase to the dispersion solvent is 1:10 (40 ml of dispersion solvent PVA) and the dispersion solvent was not previously added with EF.

<5-2> Measurement of the Yield of a Polymeric Microsphere

The yield of the polymeric microsphere obtained from Example 5-1 was measured. The measurement was carried out in the same manner as that described in Example 4-2.

As a result, as noted in Table 10, it was found that under all conditions, there is no problem in the yield of the polymeric microsphere.

TABLE 10
Yield of the prepared polymeric microsphere

| | | | |
|---|---|---|---|
| First step | Amount of | Amount of EF | |
| at room | dispersion | previously added | |
| temperature | solvent | to dispersion | Yield (%) |

| (RT) | PVA (ml) | solvent (ml) | #1 | #2 |
|---|---|---|---|---|
| | 20 | 0 | 79.56 | 70.24 |
| | | 1 | 66.48 | 63.88 |
| | | 2 | 76.68 | 66.52 |
| | 30 | 0 | 69.96 | 70.24 |
| | | 1 | 40.04 | 75.76 |
| | | 2 | 77.56 | 74.64 |
| | | 3 | 88.28 | 74.16 |
| | 40 | 0 | 67.68 | 72.4 |
| | | 1 | 67.28 | 82.8 |
| | | 2 | 39.6 | 82.2 |
| | | 3 | 77.04 | 79.64 |
| | | 4 | 55.96 | 77.92 |

<5-3> Measurement (GPC) of a Molecular Weight Change of 4.5A PLGA Before/after the Preparation of a Polymeric Microsphere

In the preparation process of the polymeric microsphere in Example 5-1, in order to determine a molecular weight change of PLGA, the molecular weight of 4.5A PLGA before/after the preparation of the polymeric microsphere was measured through gel permeation chromatography (GPC).

In the GPC, Agilent 1100 (Agilent Technologies, Inc, US) was used, and as a mobile phase, tetrahydrofuran was used. As an analysis column, PLgel 5 um 100 Å 79911GP-504 (Agilent) was used, and the flow rate was maintained at 1 ml/min. In order to obtain a standard curve, a standard product (Polymer Laboratories, molecular weight: 3390000, 1290000, 426600, 151700, 72200, 28500, 9860, 4950, 1300) was used. As the analysis sample, 8 mg of a polymeric microsphere was used, which was dissolved in 2 ml of tetrahydrofuran and filtered. The amount injected into the GPC was made 50 ul.

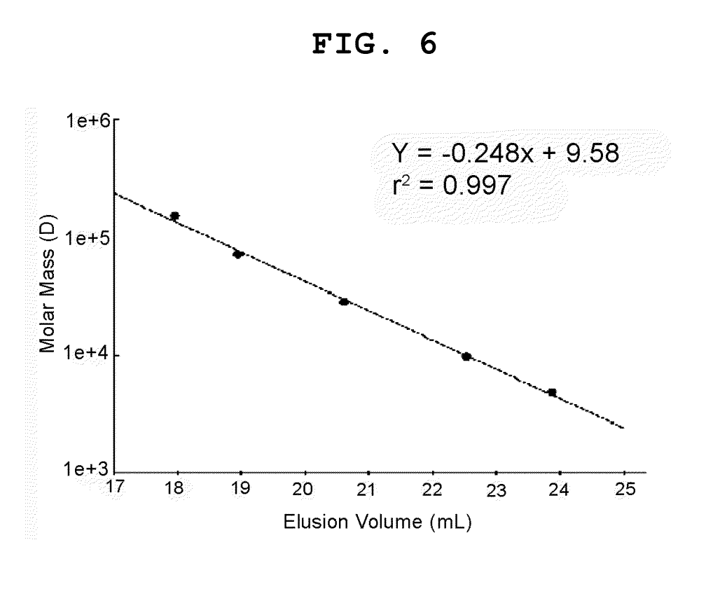

By using the standard product, the standard curve shown in FIG. 6 was obtained. Based on this, the molecular weights of 4.5A PLGA before the preparation, and 4.5A PLGA of the prepared polymeric microsphere were measured. As a result, as noted in Table 11, the molecular weight of 4.5A PLGA of the polymeric microsphere prepared by the inventive method ranged from 55421 to 58409. Thus, there was no significant change compared to the before-preparation molecular weight (57542) of 4.5A.

Accordingly, it was found that in the inventive polymeric microsphere preparation method, the polymer of 4.5A PLGA is not decomposed.

TABLE 11
Comparison of molecular weights of 4.5A PLGA

| First step | Amount of | Amount of EF | | |
|---|---|---|---|---|
| at room | dispersion | previously added | | |
| temperature | solvent | to dispersion | GPC | |
| (RT) | PVA (ml) | solvent (ml) | Mw | polydispersity |
| | 20 | 0 | 57901 | 1.4594 |
| | | 1 | 57294 | 1.5004 |
| | | 2 | 55743 | 1.5865 |
| | 30 | 0 | 57498 | 1.5405 |
| | | 1 | 55573 | 1.6392 |
| | | 2 | 57486 | 1.5552 |
| | | 3 | 55421 | 1.649 |
| | 40 | 0 | 57788 | 1.4495 |
| | | 1 | 57704 | 1.5207 |
| | | 2 | 57994 | 1.5454 |
| | | 3 | 57423 | 1.4922 |
| | | 4 | 58409 | 1.4399 |

| | | |
|---|---|---|
| Before preparation of | 57542 | 1.6122 |
| microsphere, 4.5A PLGA | | |

### Example 6

### Measurement of a Molecular Weight Change of a Polymer Compound of a Drug-Containing Polymeric Microsphere

It was found that in Example 5-3, when a drug is not contained, there is no change in the molecular weight of the polymeric microsphere. Then, a polymeric microsphere containing a drug was prepared under various conditions, and the molecular weight of the polymer compound was measured.

TABLE 12
Preparation conditions of a polymeric microsphere

| | | Amount | | | | Amount | | |
|---|---|---|---|---|---|---|---|---|
| | Kind | of | | Content | | of | Temperature | Temperature |
| Test | of | polymer | Kind | of | Organic | organic | in | in second |
| no. | polymer | (mg) | of a drug | a drug | solvent | solvent | first step | step |
| 1 | 5050 | 1000 | Anastrozole | 111 mg | Ethyl | 27 | 33 | 40 |
| | 2A | | | | formate | | | |
| 2 | 7525 | 1000 | Anastrozole | 667 mg | Ethyl | 19 | 4 | 33 |
| | 2.5E | | | | formate | | | |
| 3 | 6535 | 250 | olanzapine | 60 mg | Ethyl | 4 | 4 | 40 |
| | 4.5A | | | | formate | | | |

A dispersed phase was prepared according to Table 12, and was emulsified in 0.5% PVA so as to provide an emulsion. Then, 10N NaOH solution was added to the emulsion, followed by a reaction for 30 minutes. The resultant product was added with distilled water, and a polymeric microsphere was filtered and separated. The separated polymeric microsphere was re-dispersed in 0.1% PVA solution, followed by vacuum-drying. In the preparation of the polymeric microsphere, the first step for adding a base or acid solution and carrying out the reaction was carried out, and after the reaction, the second step for re-dispersing the polymeric microsphere filtered and separated in a PVA solution and stirring the mixture was carried out. The temperatures of the respective steps were maintained as noted in Table 12.

The molecular weight of the polymer compound of the prepared polymeric microsphere was measured in the same manner as that described in Example 5-3.

As a result, as noted in Table 13, it was found that the molecular weights of the polymer compounds of all polymeric microspheres can be sufficiently maintained. Thus, it was determined that in the inventive preparation method, a molecular weight of a polymer compound can be well maintained irrespective of conditions such as the kind of a drug, the reaction temperature.

TABLE 13
Measurement result of a molecular weight of a polymer
compound before/after polymeric microsphere preparation

| Test | Molecular weight before | Molecular weight after |
|---|---|---|
| No | microsphere preparation | microsphere preparation |
| 1 | 15000 | 15709 |
| 2 | 29013 | 27737 |
| 3 | 56694 | 54116 |

### Example 7

### Preparation of a Polymeric Microsphere Containing Olanzapine

<7-1> Preparation of a Polymeric Microsphere Containing Olanzapine

A polymeric microsphere containing olanzapine was prepared under the condition noted in Table 14 according to the inventive polymeric microsphere preparation method.

60 mg of olanzapine and 250 mg of 6535 4.5A PLGA were dissolved in 4 ml of ethyl formate. The dispersed phase was emulsified in a dispersion solvent (0.5% PVA) previously added with ethyl formate according to Table 14, so as to provide an emulsion. Then, 10N NaOH solution was added to the emulsion, followed by a reaction for 30 minutes. The resultant product was added with distilled water, and a polymeric microsphere was filtered and separated. The separated polymeric microsphere was re-dispersed in 0.1% PVA solution, followed by vacuum-drying. In the preparation of the polymeric microsphere, the first step for adding a base or acid solution and carrying out the reaction was carried out at room temperature, and after the reaction, the second step for re-dispersing the polymeric microsphere filtered and separated in a PVA solution and stirring the mixture was carried out at 40° C.

TABLE 14
Preparation conditions of a polymeric microsphere containing olanzapine

| | | | Amount of | | | |
|---|---|---|---|---|---|---|
| | | Amount | EF | | | |
| | | of | previously | | | |
| | | dispersion | added to | | Temperature | |
| Temperature | | solvent | dispersion | | of | NaOH for |
| of | Kind | PVA | solvent | olanzapine | second | Solvent |
| first step | of PLGA | (ml) | (ml) | (mg) | step | decomposition |
| Room | 6535DLG | 30 | 2 | 60 | 40 | 7.4 |
| temperature | 4.5A | | 3 | 60 | 40 | 8.6 |
| (RT) | | 40 | 3 | 60 | 40 | 8.4 |
| | | | 4 | 60 | 40 | 9.6 |

<7-2> Measurement of the Amount of a Remaining Solvent, the Loading Efficiency of Olanzapine, and the Yield of a Polymeric Microsphere

The amount of the remaining solvent of the polymeric microsphere prepared from Example 7-1 was measured in the same manner as that described in Example 1.

The prepared polymeric microsphere was placed on a dried vessel, and its weight after freeze-drying was measured. Then, the yield of the polymeric microsphere was calculated by the equation below.

Yield(%)=(weight of polymeric microsphere)/(sum of weights of polymer and drug used in the preparation)×100

In the loading efficiency of olanzapine, the loading amount and the loading efficiency of the prepared polymeric microsphere were measured as described below.

10 mg of a dried polymeric microsphere was dissolved in a solution of acetonitrile: water=50:50 (v/v), and was diluted a proper number of times. The resultant solution was filtered, and analyzed by UPLC (Ultra Performance Liquid Chromatography). Then, the loading amount and the loading efficiency were calculated. As an UPLC device, ACQUITY (Waters, Germany) was used, and as a column, HSS C18 (Waters ACQUITY UPLC, Japan) was used. A mobile phase included ammonium acetate buffer and acetonitrile in a mixed ratio of 50:50, and a dilution solution included acetonitrile and water in a mixed ratio of 50:50.

Loading amount(%)=(weight of detected drug/weight of microsphere)×100

Loading efficiency(%)=loading amount/(theoretical loading amount)×100

theoretical loading amount(%)=(weight of drug used in the preparation)/(sum of weights of polymer and drug used in the preparation)×100

As a result, it was found that the small amount of the remaining solvent was maintained as noted in Table 15, and almost all the loading efficiencies and the yields showed a high value of 70% or more as noted in Table 16.

TABLE 15
Amount of a remaining solvent of a
prepared polymeric microsphere

| | | Amount | | | | |
|---|---|---|---|---|---|---|
| | | of EF | | | | |
| | Amount | previously | | | | |
| | of | added | | | | |
| Temperature | dispersion | to | | | | |
| of | solvent | dispersion | Remaining | | Remaining | |
| first | PVA | solvent | EF (%) | | ethanol (%) | |
| step | (ml) | (ml) | #1 | #2 | #1 | #2 |
| Room | 30 | 2 | 0.023 | 0.006 | 0.199 | 0.163 |
| temperature | | 3 | 0.013 | 0.003 | 0.168 | 0.116 |
| (RT) | 40 | 3 | 0.022 | 0.011 | 0.208 | 0.140 |
| | | 4 | 0.013 | 0.014 | 0.201 | 0.145 |

TABLE 16
Loading efficiency and yield of the
prepared polymeric microsphere

| | | Amount | | | | |
|---|---|---|---|---|---|---|
| | | of EF | | | | |
| | Amount | previously | | | | |
| Temp- | of | added | | | | |
| erature | dispersion | to | Loading | | | |
| in | solvent | dispersion | efficiency | | | |
| first | PVA | solvent | (%) | | Yield (%) | |
| step | (ml) | (ml) | #1 | #2 | #1 | #2 |
| Room | 30 | 2 | 80.719 | 76.789 | 73.613 | 72.616 |
| temp- | | 3 | 80.215 | 69.635 | 74.226 | 72.958 |
| erature | 40 | 3 | 80.858 | 74.364 | 71.613 | 75.723 |
| (RT) | | 4 | 74.205 | 68.040 | 72.613 | 75.942 |

### Example 8

### Preparation of an Olanzapine-Containing Polymeric Microsphere by Using Ethyl Acetate as a Water-Insoluble Organic Solvent

<8-1> Preparation of an Olanzapine-Containing Polymeric Microsphere

An olanzapine-containing polymeric microsphere was prepared by using another water-insoluble organic solvent than the water-insoluble organic solvent (ethyl formate) used in Example 7.

In this Example, as the water-insoluble organic solvent, ethyl acetate (EA) was used, and the polymeric microsphere was prepared under the preparation conditions noted in Table 17.

60 mg of olanzapine and 250 mg of 4.5A PLGA were dissolved in 4 ml of ethyl acetate so as to provide a dispersed phase. The dispersed phase was emulsified in a dispersion solvent (0.5% PVA) previously added with ethyl acetate according to Table 17 so as to provide an emulsion. Then, 10N NaOH solution was added to the emulsion, followed by a reaction for 30 minutes. The resultant product was added with distilled water, and a polymeric microsphere was filtered and separated. The separated polymeric microsphere was re-dispersed in 0.1% PVA solution, followed by vacuum-drying. In the preparation of the polymeric microsphere, the first step for adding a base or acid solution and carrying out the reaction was carried out at 4° C., and after the reaction, the second step for re-dispersing the polymeric microsphere filtered and separated in a PVA solution and stirring the mixture was carried out at 40° C.

TABLE 17
Preparation conditions of a polymeric microsphere containing olanzapine

| | | | Amount | | | |
|---|---|---|---|---|---|---|
| | | | of EA | | | |
| | | Amount | previously | | | |
| | | of | added | | | |
| Temperature | | dispersion | to | | Temperature | NaOH |
| of | | solvent | dispersion | | of | (ml) for |
| first | Kind | PVA | solvent | olanzapine | second | Solvent |
| step | of PLGA | (ml) | (ml) | (mg) | step | decomposition |
| 4 | 6535 | 30 | 2 | 60 | 40 | 7.4 |
| | 4.5A | | 3 | 60 | 40 | 8.6 |
| | | 40 | 3 | 60 | 40 | 8.4 |
| | | | 4 | 60 | 40 | 9.6 |

<8-2> Measurement of the Amount of a Remaining Solvent, the Loading Efficiency of Olanzapine, and the Yield of a Polymeric Microsphere

The amount of the remaining solvent of the polymeric microsphere prepared from Example 8-1 was measured in the same manner as that described in Example 1.

As a result, it was found that the small amount of the remaining solvent was maintained as noted in Table 18, and almost all the loading efficiencies and the yields showed high value of 85% or more and 70% or more, respectively, as noted in Table 19.

TABLE 18
Amount of a remaining solvent of a
prepared polymeric microsphere

| | | Amount | | | | |
|---|---|---|---|---|---|---|
| | | of EF | | | | |
| | Amount | previously | | | | |
| | of | added | | | | |
| Temperature | dispersion | to | | | | |
| of | solvent | dispersion | Remaining | | Remaining | |
| first | PVA | solvent | EA (%) | | ethanol (%) | |
| step | (ml) | (ml) | #1 | #2 | #1 | #2 |
| 4 | 30 | 2 | 0.151 | 0.153 | 0.513 | 0.410 |
| | | 3 | 0.134 | 0.120 | 0.578 | 0.506 |
| | 40 | 3 | 0.085 | 0.091 | 0.280 | 0.242 |
| | | 4 | 0.129 | 0.130 | 0.310 | 0.458 |

TABLE 19
Loading efficiency and yield of the
prepared polymeric microsphere

| | Amount of | Amount of EF | | |
|---|---|---|---|---|
| Temperature | dispersion | previously added | Loading | |
| of first | solvent | to dispersion | efficiency | Yield |
| step | PVA (ml) | solvent (ml) | (%) | (%) |
| 4 | 30 | 2 | 90.952 | 71.05 |
| | | 3 | 88.892 | 71.45 |
| | 40 | 3 | 85.335 | 75.71 |
| | | 4 | 88.993 | 67.32 |

### Example 9

### A Pharmacokinetic Test on an Olanzapine-Containing Polymeric Microsphere

<9-1> Preparation of an Olanzapine-Containing Polymeric Microsphere

According to compositions noted in Table 20, various PLGAs were used so as to prepare an olanzapine-containing polymeric microsphere.

PLGA and olanzapine were dissolved in an organic solvent (ethyl formate) so as to provide a dispersed phase. The ratio of the dispersed phase to a dispersion solvent was 1:5, and as the dispersion solvent, a 10:1 mixture of 0.5% PVA and ethyl formate was used. The dispersed phase was emulsified in the dispersion solvent cooled to 4° C., so as to provide an emulsion. Then, 10M NaOH was added to the emulsion as noted in Table 20, followed by a reaction for 30 minutes so as to provide a polymeric microsphere. The prepared polymeric microsphere was washed with distilled water, and re-dispersed in 0.1% PVA solution at 40° C., followed by stirring. Then, the polymeric microsphere was separated and vacuum-dried.

The state of the prepared polymeric microsphere was observed by an electron microscope (SEM, Hitachi S-3000N, Japan, HV 20 kv, Working Distance 15 mm, Beam current 40).

TABLE 20
Composition of an olanzapine-containing polymeric microsphere

| | | | Organic | | | |
|---|---|---|---|---|---|---|
| Preparation | | | solvent | olanzapine | Amount | 0.5% |
| No. | Kind of PLGA | | (ml) | (mg) | or NaOH | PVA |
| 1 | 8515 | 4.5E | 30 | 667 | 56 | 150 |
| 2 | 100 DL | 2E | 30 | 667 | 56 | 150 |
| 3 | 100 DL | 4.5E | 30 | 667 | 56 | 150 |
| 4 | 100 DL | 2E | 10 | 240 | 18.6 | 50 |

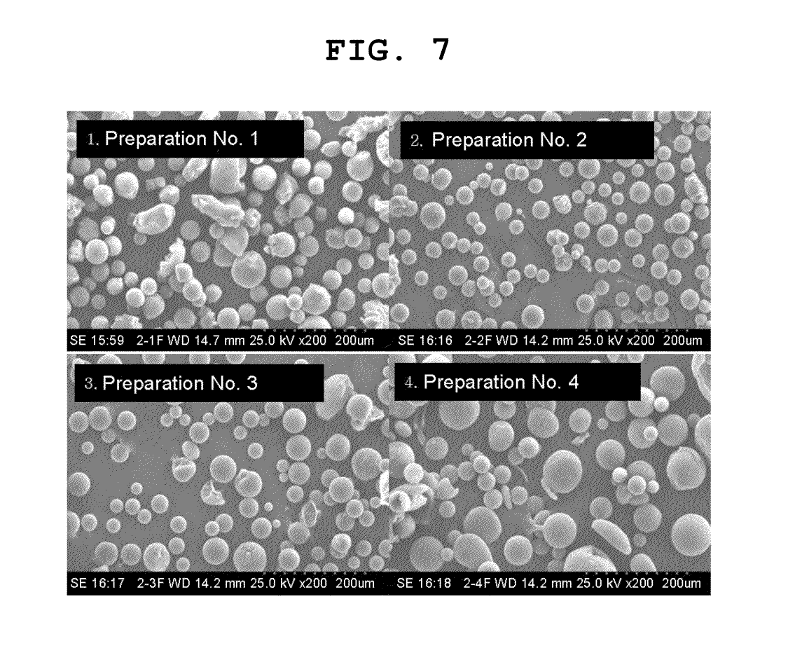

Through the observation by the electron microscope, it was found that olanzapine was well loaded into the polymeric microsphere, and all of the polymeric microspheres were well prepared in a spherical form (see FIG. 7).

<9-2> Test on Injectability and Dispersibility

The olanzapine-containing polymeric microsphere prepared from Example 9-1 was suspended at a concentration of 30% (w/v) in a saline solution containing carboxymethyl cellulose (CMC) and tween 20.1 ml of the resultant solution was loaded into a syringe (19 G) and then injected so as to determine the injectability.

As a result, as noted in Table 21, it was found that the inventive olanzapine-containing polymeric microsphere was good in injectability and dispersibility.

TABLE 21
Test result of injectability and dispersibility

| Preparation | |
|---|---|
| No | Test result |
| 1 | + |
| 2 | + |
| 3 | + |

<9-3> Test on the Persistence of an Olanzapine-Containing Polymeric Microsphere

The olanzapine-containing polymeric microsphere prepared from Example 9-1 was suspended in an excipient solution (saline including carboxymethyl cellulose and tween 20), and was administered to female SD rats (aged 9 weeks) in an amount of 40 mg/kg through intramuscular injection. Then, at a predetermined time interval, the blood was collected, and the concentration of olanzapine in the blood was measured.

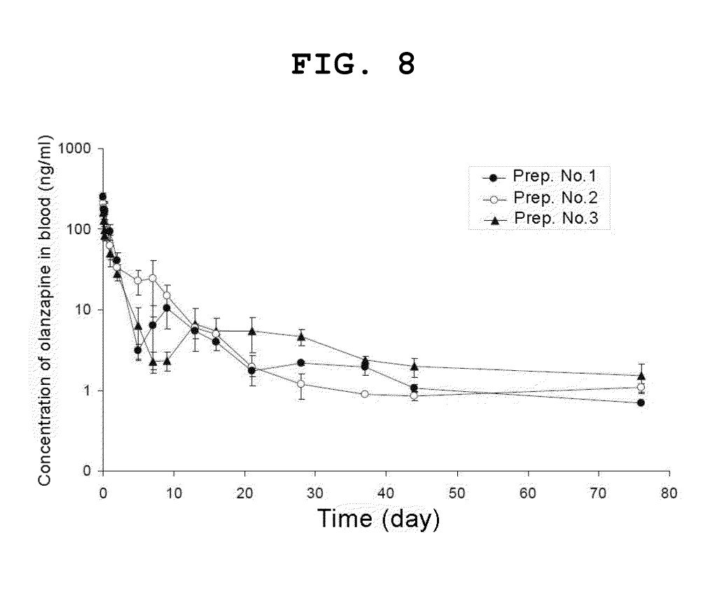

As a result, as shown in FIG. 8, it was found that the inventive olanzapine-containing polymeric microsphere released olanzapine for a maximum of 80 days.

### Example 10

### Preparation of an Anastrozole-Containing Polymeric Microsphere

According to the inventive polymeric microsphere preparation method, a polymeric microsphere was prepared by using another kind of a polymer, another drug, and another loading amount than those used in the above described Example.

<10-1> Preparation of an Anastrozole-Containing Polymeric Microsphere

According to the formulation noted in Table 22, under various conditions, an anastrozole-containing polymeric microsphere was prepared.

TABLE 22
Composition of an anastrozole-containing polymeric microsphere

| Preparation | | |
|---|---|---|
| No. | Kind of PLGA | anastrozole (mg) |
| 1 | 5050-4A | 429 |
| 2 | 7525-2.5E | 429 |
| 3 | 7525-7E | 429 |
| 4 | 8515-3E | 429 |
| 5 | 8515-4.5E | 429 |
| 6 | 100-DL 2E | 429 |
| 7 | 7525-2.5E | 667 |
| 8 | 8515-4.5E | 667 |

Anastrozole and 1000 mg of PLGA were completely dissolved in 19 ml of ethyl formate so as to provide a dispersed phase. The dispersed phase was emulsified in 0.5% PVA (dispersion solvent) which was cooled at 4° C. and added with 8 ml of ethyl formate, followed by stirring so as to provide an emulsion. Then, 34 ml of 10M. NaOH was added to the emulsion, followed by stirring for 30 minutes. The resultant prepared polymeric microsphere was washed with distilled water and re-dispersed in 0.1% PVA at 33° C., followed by stirring. Then, the resultant product was filtered and freeze-dried so as to provide a polymeric microsphere.

<10-2> Test on Basic Characteristics of a Prepared Anastrozole-Containing Polymeric Microsphere

The prepared polymeric microsphere was placed on a dried vessel, and then its weight after freeze-drying was measured. Then, the yield of the polymeric microsphere was calculated by the equation below.

Yield(%)=(weight of polymeric microsphere)/(sum of weights of polymer and drug used in the preparation)×100

The loading amount and the loading efficiency of the prepared polymeric microsphere were measured as described below.

10 mg of a dried polymeric microsphere was dissolved in a solution of acetonitrile: water=50:50 (v/v), and was diluted a proper number of times. The resultant solution was filtered, and analyzed by UPLC. Then, the loading amount and the loading efficiency were calculated. As an UPLC device, ACQUITY (Waters, Germany) was used, and as a column, HSS C18 (Waters ACQUITY UPLC, Japan) was used. A mobile phase included ammonium acetate buffer and acetonitrile in a mixed ratio of 50:50, and a dilution solution included acetonitrile and water in a mixed ratio of 50:50.

Loading amount(%)=(weight of detected drug/weight of microsphere)×100

Loading efficiency(%)=loading amount/(theoretical loading amount)×100

theoretical loading amount(%)=(weight of drug used in the preparation)/(sum of weights of polymer and drug used in the preparation)×100

Particle size analysis was carried out by using Malvern mastersizer (Malvern Instruments Ltd, UK).

The amount of the remaining solvent was measured in the same manner as that described in Example 1, and the shape of the polymeric microsphere was photographed in the same manner as that described in Example 2-2.

TABLE 23
The yield, the loading amount, and the concentration of a remaining
solvent of the prepared polymeric microsphere

| | | Loading | Loading | Average | Moisture | remaining | Remaining |
|---|---|---|---|---|---|---|---|
| Preparation | yield | amount | efficiency | size | content | EF | ethanol |
| No. | (%) | (%) | (%) | (um) | (%) | (%) | (%) |
| 1 | 82.44 | 26.6 | 88.68 | 56.0 | — | N.D. | N.D. |
| 2 | 82.93 | 24.68 | 82.25 | 44.8 | 0.47 | N.D. | N.D. |
| 3 | 98.81 | 25.41 | 84.71 | 61.6 | — | 0.026 | N.D. |
| 4 | 83.55 | 25.88 | 86.26 | 49.9 | 0.46 | N.D. | N.D. |
| 5 | 85.86 | 26.41 | 87.12 | 59.8 | 0.51 | N.D. | N.D. |
| 6 | 81.88 | 25.00 | 83.34 | 41.1 | 0.47 | N.D. | N.D. |
| 7 | 79.48 | 30.41 | 76.04 | 32.5 | — | N.D. | N.D. |
| 8 | 82.42 | 32.97 | 82.42 | 52.7 | — | N.D. | N.D. |

As a result, as noted in Table 23, the yield and the loading efficiency were almost 80% or more, which is appropriate irrespective of the kind of PLGA. Also, the average particle size was found to have an appropriate value ranging from 30 to 60 um. The moisture content was about 0.5%, and the remaining solvent was included in a very low amount, which was found to be appropriate irrespective of the kind of a test sample.

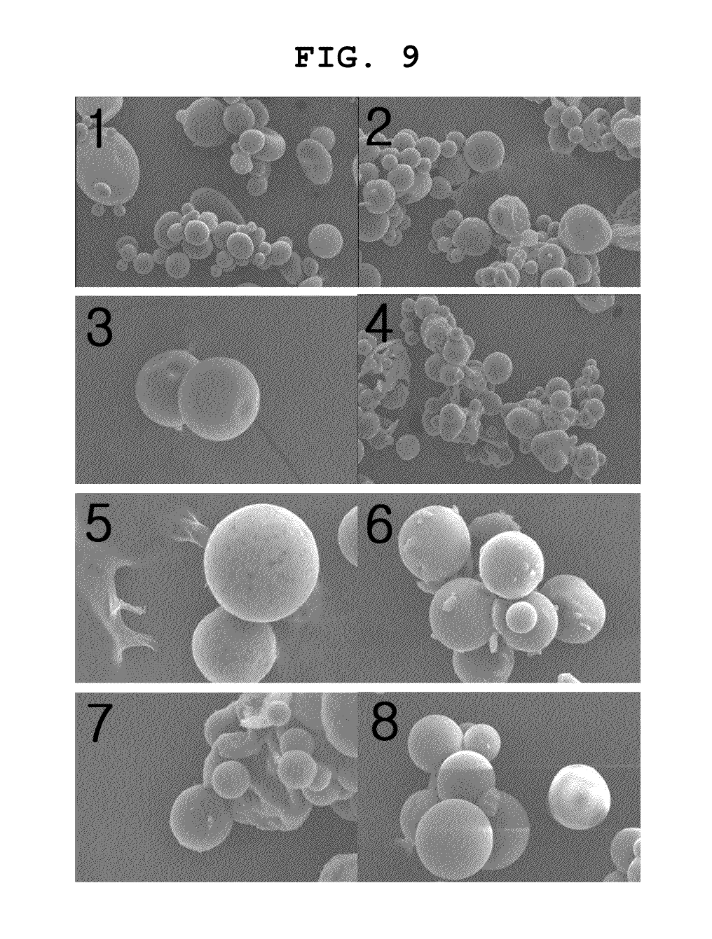

Also, through the photograph by the electron microscope, as shown in FIG. 9, it can be found that the polymeric microsphere was generally well prepared in a smooth spherical shape irrespective of the kind of used PLGA. Especially, it was found that even when the drug content was increased up to 40%, the drug was well loaded and no crystallization of the drug was observed from the surface.

<10-3> Test on the Stability of a Prepared Anastrozole-Containing Polymeric Microsphere

The analysis on a related compound within a prepared polymeric microsphere was carried out for Preparation No. 8. A polymeric microsphere was weighted in such a manner that API (Active Pharmaceutical Ingredients) anastrozole can be 250 ug/ml with respect to the loading amount within the polymeric microsphere, and then was diluted in a solution of acetonitrile: DW=50:50. Then, it was analyzed by UPLC.

For GPC analysis, 30 mg of the prepared polymeric microsphere was dissolved in 3 ml of (o-CP: chloroform=1:3), and was subjected to GPC analysis by an RI detector through a column of PLgel 5 μm Mixed-D*2ea at 40° C. at a rate of 0.7 ml/min.

For a test of in vivo stability simulation, a polymeric microsphere (Preparation No. 8) in a tube was introduced in a desicator having water at the bottom thereof, with a humidity of 100%, and then was put in a 40° C. stability tester. In a predetermined interval (4 weeks, 2 months), a sample (n=3) was collected, dissolved in acetonitrile, and diluted with methanol. Then, the change of a drug content and a related compound was analyzed.

As a result, as noted in Table 24, the polymeric microsphere of the present invention was found to have a related compound of an average value of 0.17%. In general, a related compound is regulated in such a manner that a total of related compounds are 1% or less with respect to a drug raw material, and an individual related compound is 0.1% or less. Thus, it was determined that in the inventive polymeric microsphere preparation process, the stability was achieved.

TABLE 24
Analysis result of a related compound in a polymeric microsphere

| Preparation | Total of related | Average of total of |
|---|---|---|
| no | compounds (%) | related compounds (%) |
| 8 | 0.18 | 0.17 |
| | 0.15 | |
| | 0.17 | |

Also, through GPC analysis, the molecular weight of PLGA before/after the preparation was measured. As a result, as noted in Table 25, it was found that most polymers show no change in their molecular weights.

TABLE 25
Measurement result of a molecular weight
of a polymer used in the preparation

| | | Molecular |
|---|---|---|
| Preparation | Molecular | weight |
| no. | weight (Mw) | preservation (%) |
| 1 | 38,000 | 65.5 |
| 2 | 24,412 | 84.2 |
| 3 | 79,870 | 68.3 |
| 4 | 37,026 | 94.9 |
| 5 | 69,843 | 105.7 |
| 6 | 23,937 | 108.3 |
| 7 | 27,737 | 95.6 |
| 8 | 73,065 | 110.5 |

As a result of in vivo stability simulation, as noted in Table 26, the average content (%) of a total of related compounds in respective periods during the entire period was 0.23 or less, and a change of a drug content was less than 10%. Thus, it was determined that the inventive preparation method shows a high stability.

TABLE 26
Result of in vivo stability simulation

| | Total of | | |
|---|---|---|---|
| | related | Drug | relative |
| Period | compounds (%) | content % | content % |
| Start | 0.17 | 33.341.19 | 100.0 |
| 4 weeks | 0.12 | 33.951.31 | 101.8 |
| 2 months | 0.23 | 30.220.14 | 90.6 |

<10-4> Test 1 on Persistence of a Prepared Anastrozole-Containing Polymeric Microsphere—In Vitro Release Test

In order to determine the persistence of drug release of a polymeric microsphere prepared by the inventive method, in vitro drug release test was carried out.

In a dialysis membrane (MWCO=12 to 14 KD), a polymeric microsphere was introduced. Then, the dialysis membrane was filled with PBS, and was placed in PBS with temperature equilibrium, followed by continuous stirring at a 37° C. thermostat. Then, in a predetermined time interval, the amount of released drug was measured. When the drug release is completed, the remaining polymeric microsphere was vacuum-dried, and then, the amount of remaining drug was measured.

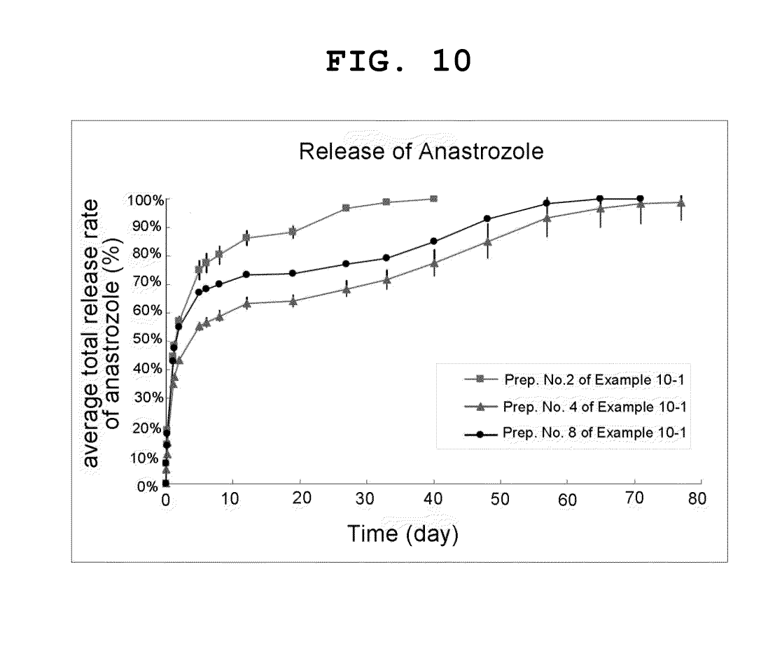

As a result, as shown in FIG. 10, it was determined that after the drug release, polymeric microspheres of all formulations hardly have remaining drugs. Also, according to the kinds of the polymer compounds used in the preparation, as the ratio of lactide was increased, the drug was slowly released. Meanwhile, it was determined that as a drug content was increased, the release rate was increased. Thus, it was determined that it is possible to adjust the release rate by adjusting the kind of the polymer compound used in the polymeric microsphere preparation, and the amount of the loaded drug.

<10-5> Test 2 on Persistence of a Prepared Anastrozole-Containing Polymeric Microsphere—Animal Test

In order to determine injectability and dispersibility, a prepared polymeric microsphere was added with a saline solution containing carboxymethyl cellulose (CMC) and tween 20, so as to provide a suspension. The suspension was loaded into a syringe (19 G or 20 G) and then injected in an amount of 1 ml so as to determine the injectability. The result was determined at a concentration of 10% of polymeric microsphere/suspension (w/v).

When a suspension was administered to a rat through injection, each preparation was suspended in an excipient solution (saline including CMC and tween 20) before injection. Female SD rats (aged 9 weeks) were used, and the suspension was administered in an amount of 20 mg/kg through intramuscular injection. Then, at a predetermined time interval, the blood was collected, and the concentration of anastrozole in the blood was measured.

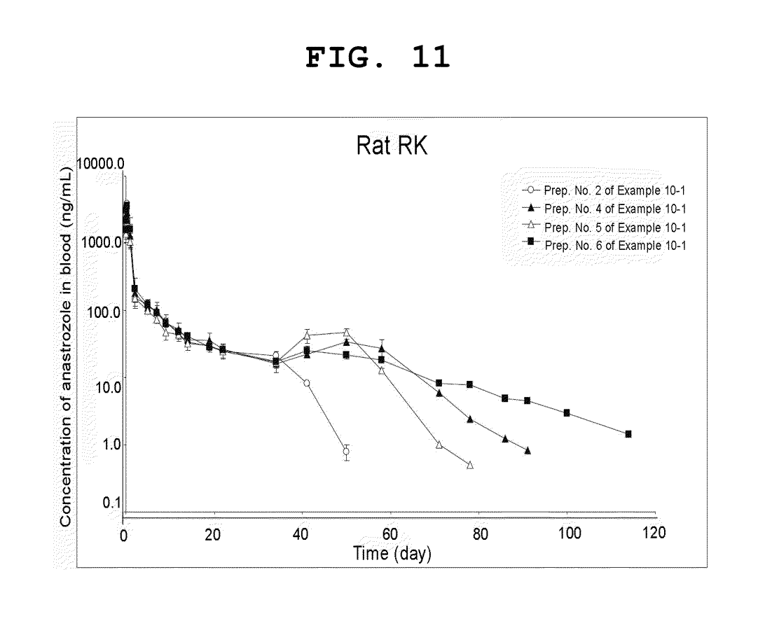

As a result, as shown in FIG. 11, the polymeric microsphere prepared by the inventive method was found to release a drug for a maximum of 120 days.

As described above, the present invention provides a novel a method for preparing microsphere [symbol] and microspheres produced by thereby. More particularly, the present invention relates to a method for preparing a polymeric microsphere comprising the steps of: mixing a water-insoluble organic solvent with a dispersion solvent; mixing a polymer compound, a drug and a water-insoluble organic solvent so as to prepare a dispersed phase; mixing the dispersed phase with the dispersion solvent mixed with the water-insoluble organic solvent so as to prepare an emulsion; and adding a base or an acid to the prepared emulsion so as to remove the water-insoluble organic solvent from the emulsion and a polymeric microsphere thereby, and a composition for drug delivery comprising the microspheres. In the inventive preparation method, it is possible to conveniently prepare a drug-containing polymeric microsphere within a short time using a small amount of water, with the generation of a minimized amount of waste solution without a requirement of a conventional solvent-evaporation or solvent-extraction process. Also, since a low concentration of remaining solvent within a prepared polymeric microsphere can be maintained, it is effective in preparation in sustained-release type medicines.

1. A method for preparing a polymeric microsphere, comprising the steps of:
(a) mixing a water-insoluble organic solvent with a dispersion solvent;
(b) mixing a polymer compound, a drug and a water-insoluble organic solvent so as to prepare a dispersed phase;
(c) mixing the dispersed phase of step (b) with the dispersion solvent mixed with the water-insoluble organic solvent of step (a) so as to prepare an O/W (oil-in-water), O/O (oil-in-oil) or W/O/W (water-in oil-in-water) type emulsion; and
(d) adding a base or an acid to the emulsion of step (c) so as to remove the water-insoluble organic solvent from the emulsion.

2. A method for preparing a polymeric microsphere, comprising the steps of:
(a) mixing a water-insoluble organic solvent with a dispersion solvent;
(b) mixing a polymer compound, a drug and a water-insoluble organic solvent so as to prepare a dispersed phase;
(c) mixing the dispersed phase of step (b) with the dispersion solvent mixed with the water-insoluble organic solvent of step (a) so as to prepare an O/W (oil-in-water), O/O (oil-in-oil) or W/O/W (water-in oil-in-water) type emulsion; and
(d) adding a base or an acid to the prepared emulsion of step (c) so as to remove the water-insoluble organic solvent from the emulsion.
(e) obtaining the polymeric microsphere prepared of step (d) and re-dispersing the obtained polymeric microsphere in a warmed dispersion solvent.

3. The method of claim 1 or 2, wherein the water-insoluble organic solvent of step (b) is identical to the water-insoluble organic solvent of step (a).

4. The method of claim 1 or 2, wherein the step (c) is characterized that ratio of the dispersed phase of step (b) and the dispersion solvent of step (a) which is mixed with water-insoluble organic solvent is 1:3 to 100.

5. The method of claim 1 or 2, wherein the step (c) is characterized that ratio of the dispersed phase of the step (b) and the dispersion solvent of the of step (a) which is mixed with water-insoluble organic solvent is 1:4 to 20.

6. The method of claim 2, wherein the temperature of the heated dispersion solvent is 20° C. to 80° C.

7. The method of claim 2, wherein the temperature of the heated dispersion solvent is 30° C. to 50° C.

8. The method of claim 1 or 2, wherein the temperature of the emulsion of the step (c) and the step (d) is 0° C. to 35° C.

9. The method of claim 1 or 2, wherein the polymer compound is selected from the group consisting polylactic acid, polylactide, polylactic-co-glycolic acid, polylactide-co-glycolide (PLGA), polyphosphazene, polyiminocarbonate, polyphosphoester, polyanhydride, polyorthoester, lactic acid-caprolactone copolymer, polycaprolactone, polyhydroxyvalerate, polyhydroxybutyrate, polyamino acid, lactic acid-amino acid copolymer, and a mixture thereof.

10. The method of claim 1 or 2, wherein the polymer compound is polylactide-co-glycolide (PLGA).

11. The method of claim 1 or 2, wherein drug is/are one or more selected from the group consisting of progesterone, haloperidol, thiothixene, olanzapine, clozapine, bromperidol, pimozide, risperidone, ziprasidone, diazepam, ethyl loflazepate, alprazolam, nemonapride, fluoxetine, sertraline, venlafaxine, donepezil, tacrine, galantamine, rivastigmine, selegiline, ropinirole, pergolide, trihexyphenidyl, bromocriptine, benztropine, colchicine, nordazepam, etizolam, bromazepam, clotiazepam, mexazolum, buspirone, goserelin acetate, somatotropin, leuprolide acetate, octreotide, cetrorelix, sandostatin acetate, gonadotropin, fluconazole, itraconazole, mizoribine, cyclosporin, tacrolimus, naloxone, naltrexone, cladribine, chlorambucil, tretinoin, carmustine, anagrelide, doxorubicin, anastrozole, idarubicin, cisplatin, dactinomycin, docetaxel, paclitaxel, raltitrexed, epirubicin, letrozole, mefloquine, primaquine, oxybutynin, tolterodine, allylestrenol, lovostatin, simvastatin, provastatin, atrovastatin, alendronate, salcatonin, raloxifene, oxadrolone, conjugated estrogen, estradiol, estradiol valerate, estradiol benzoate, ethinyl estradiol, etonogestrel, levonorgestrel, tibolone, piroxicam and norethisterone.

12. The method of claim 1 or 2, wherein the water-insoluble organic solvent has a back bone which is selected from the group consisting of acid halogen, anhydride, phosphoric anhydride, ester, carboxylic esters, phosphoric esters, sulfuric acid esters, nitric esters, boric acid ester, amide and carboxylic amides.

13. The method of claim 1 or 2, wherein the water-insoluble organic solvent is selected from the group consisting of methyl acetate, ethyl acetate, propyl acetate, isopropyl acetate, butyl acetate, methyl formate, ethyl formate, isopropyl formate, propyl formate, butyl formate, methyl dichloroacetate, methyl chloroacetate, ethyl chloroacetate, ethyl dichloroacetate, methyl fluoroacetate, methyl difluoroacetate, ethyl fluoroacetate, ethyl difluoroacetate, maleic anhydride, acetic anhydride, propionic anhydride, phosphoric anhydride, acetamide, propionamide, butylamide and carboxyl amide.

14. The method of claim 1 or 2, wherein the dispersion solvent is polyvinyl alcohol solution or polysorbates solution or a co-solvent thereof, or a non-aqueous dispersion solvent which is selected from the group consisting of silicone oil containing lipophilic emulsifier such as glycerin esters of fatty acids or lecithin, vegetable oil, toluene, or xylene.

15. The method of claim 1 or 2, wherein the base is selected from the group consisting of sodium hydroxide (NaOH), lithium hydroxide (LiOH), potassium hydroxide (KOH), ammonium hydroxide (NH₄OH), copper hydroxide (Cu(OH)₂), and iron hydroxide (Fe(OH)₃).

16. The method of claim 1 or 2, wherein the acid is selected from the group consisting of hydrochloric acid (HCl), nitric acid (HNO₃), sulfuric acid (H₂SO₄), acetic acid (CH₃COOH), boric acid (H₃BO₃) and carbonic acid (H₂CO₃).

17. A polymeric microspheres prepared by the method of claim 1 or 2.

18. A composition for drug delivery comprising the polymeric microspheres of claim 17 as an active ingredient.
